(19) United States
(12) Patent Application Publication
(10) Pub. No.: US 2026/0263096 A1
(43) Pub. Date: Sep. 10, 2026

(54) DISTAL TIP STRUCTURES WITH SHOCKWAVE ELEMENTS FOR MEDICAL DEVICES

(71) Applicant: AURIS HEALTH, INC., Santa Clara, CA (US)

(72) Inventors: Timothy REPPERT, Felton, CA (US); Alfred ROSEN, San Mateo, CA (US); Monty LIONG, Foster City, CA (US); Elizabeth Abigail EATON, Redwood City, CA (US); Margaret SAXELBY, Milpitas, CA (US)

(73) Assignee: AURIS HEALTH, INC., Santa Clara, CA (US)

(21) Appl. No.: 19/556,015

(22) Filed: Mar. 4, 2026

Related U.S. Application Data
(60) Provisional application No. 63/766,441, filed on Mar. 4, 2025.
(60) Provisional application No. 63/872,016, filed on Aug. 28, 2025.

(51) Int. Cl. A61B 17/22

(52) U.S. Cl. CPC A61B 17/22012

(57) ABSTRACT

A catheter includes an elongate shaft having opposing proximal and distal ends, and a tip structure provided at the distal end and including an end surface at a distal end of the tip structure, an inner surface defining a lumen terminating at the distal end of the tip structure, and an outer surface opposite the inner surface. One or more shockwave elements are arranged on the inner surface and configured to propagate shockwaves to fragment an object.

### FIELD OF THE DISCLOSURE

The present disclosure relates generally to medical devices and, more particularly, to medical devices that are capable of fragmenting objects within a patient.

### BACKGROUND OF THE DISCLOSURE

Various medical procedures involve the use of one or more medical devices for accessing and fragmenting an object at a target anatomical site in a patient. In some instances, the use of devices in connection with a procedure can adversely affect the health of the patient, the integrity of the medical device(s), and/or the efficacy of the procedure.

There is a need in the art for an improved medical device that can access and fragment objects at target anatomical sites in a patient.

### BRIEF DESCRIPTION OF THE DRAWINGS

FIG. 1 illustrates an example robotic medical system arranged for a diagnostic and/or therapeutic urology procedure in accordance with one or more embodiments.

FIG. 2 illustrates an example robotic medical system arranged for a diagnostic and/or therapeutic bronchoscopy procedure in accordance with one or more embodiments.

FIG. 3 illustrates an example table-based robotic system in accordance with one or more embodiments.

FIG. 4 illustrates example medical system components that may be implemented in any of the medical systems of FIGS. 1-3 in accordance with one or more embodiments.

FIG. 5A illustrates an example catheter disposed in the kidney of a patient in accordance with one or more embodiments.

FIG. 5B is a schematic end view of a tip structure of the catheter of FIG. 5A.

FIG. 5C is a schematic cross-sectional view of the tip structure of the catheter of FIG. 5A.

FIG. 6 illustrates a perspective view of an example robotically-controllable catheter in accordance with one or more embodiments.

FIG. 7A illustrates a side view of the shaft of the catheter from FIG. 6 in accordance with one or more embodiments.

FIG. 7B illustrates an enlarged cross-sectional view of the region indicated in FIG. 7A.

FIG. 7C illustrates a cross-sectional view of the shaft of the catheter from FIG. 7B as taken along section line 7C-7C in accordance with one or more embodiments.

FIG. 8 is a schematic perspective view of a portion of a tip structure of a catheter.

FIG. 9A is a schematic cross-sectional view of a tip structure of a catheter.

FIG. 9B is a schematic end view of the tip structure shown in FIG. 9A.

FIG. 10A illustrates a schematic cross-section of a kidney to illustrate a kidney stone at a target site where the stone is difficult to reach and/or manipulate with a catheter or ureteroscope.

FIG. 10B illustrates an isometric view of an example tip structure of the catheter of FIG. 10A, according to one or more additional embodiments of the disclosure.

FIG. 10C is a schematic end view of the tip structure shown in FIG. 10B.

FIG. 10D is a schematic cross-sectional view of an alternative tip structure of the catheter of FIG. 10A showing a shockwave element arranged to generate shockwaves away from the tip structure in a generally backwards trajectory.

FIG. 11 illustrates an example embodiment of a scope.

FIG. 12 illustrates the scope of FIG. 11 navigated through an anatomical pathway of a kidney.

FIG. 13 illustrates a cross-sectional view of a kidney showing a percutaneous catheter and a ureteroscope in position to treat a kidney stone.

FIG. 14A illustrates an example probe with one or more shockwave elements inserted through a catheter disposed in the kidney of a patient in accordance with one or more embodiments.

FIG. 14B illustrates a side view of the probe shown in FIG. 14A.

FIGS. 15A and 15B are isometric and cross-sectional side views, respectively, of an example tip structure, according to one or more embodiments of the present disclosure.

FIGS. 16A and 16B are isometric and cross-sectional side views, respectively, of another example tip structure, according to one or more additional embodiments of the present disclosure.

FIGS. 17A and 17B are isometric and cross-sectional side views, respectively, of another example tip structure, according to one or more additional embodiments of the present disclosure.

FIGS. 18A and 18B are isometric and cross-sectional side views, respectively, of another example tip structure, according to one or more additional embodiments of the present disclosure.

FIG. 19 is a flow chart of an example method of fragmenting an object located at a target site within a patient, according to one or more aspects of the present disclosure.

### DETAILED DESCRIPTION

Embodiments of the present disclosure will now be described in detail with reference to the accompanying Figures. Like elements in the various figures may be denoted by like reference numerals for consistency. Further, in the following detailed description of embodiments of the present disclosure, numerous specific details are set forth in order to provide a more thorough understanding of the claimed subject matter. However, it will be apparent to one of ordinary skill in the art that the embodiments disclosed herein may be practiced without these specific details. In other instances, well-known features have not been described in detail to avoid unnecessarily complicating the description. Additionally, it will be apparent to one of ordinary skill in the art that the scale of the elements presented in the accompanying Figures may vary without departing from the scope of the present disclosure.

Embodiments in accordance with the present disclosure generally relate to medical devices and, more particularly, to medical devices that fragment (e.g., break up) objects within a patient, including both human and non-human subjects, and further including both living and non-living subjects. More specifically, the present disclosure relates to medical devices (e.g., aspiration catheters) with one or more shockwave elements configured to generate (propagate) shockwaves (e.g., ultrasonic short pulse waves) to break-up an object (e.g., kidney stones, tissue, calcification) to facilitate removal of the object from a patient.

With respect to medical devices relevant to the present disclosure, the term “device” is used according to its broad and ordinary meaning and may refer to any type of tool, instrument, assembly, system, apparatus, component, or the like. In some contexts herein, the term “instrument” may be used substantially interchangeably with the term “device.” Additionally, the terms “distal” and “proximal” as used herein refers to a location being either closer to or away from the working end of the end effector or instrument; the term “distal” being located at or near the working end of the end effector, and the term “proximal” being located away from the working end of the end effector and otherwise closer to the surgeon.

Although certain aspects of the present disclosure are described in detail herein in the context of renal, urological, and/or nephrological procedures, such as kidney stone removal/treatment procedures, it should be understood that such context is provided for convenience, and the concepts disclosed herein are applicable to any suitable medical procedures. For example, the following description is also applicable to other surgical/medical operations or medical procedures concerned with the removal of objects from a patient, including any object that can be removed from a treatment site or patient cavity (e.g., the esophagus, ureter, intestine, eye, etc.) via percutaneous and/or endoscopic access, such as, for example, gallbladder stone removal, lung (pulmonary/transthoracic) tumor biopsy, cataract removal, a bronchoscopy procedure, etc. However, as mentioned, description of the renal/urinary anatomy and associated medical issues and procedures is presented below to aid in the description of the concepts disclosed herein. Additionally, the following description is applicable to a medical procedure where an object is broken up but only partially removed from the target site or left within the target site.

Kidney stone disease, also known as urolithiasis, is a medical condition that involves the formation in the urinary tract of a solid piece of material, referred to as “urolithiases” which are also referred to as “kidney stones,” “urinary stones,” “renal calculi,” “renal lithiasis,” “nephrolithiasis,” or “bladder stones.” Urinary stones may be formed and/or found in the kidneys, the ureters, and the bladder (referred to as “bladder stones”). Such urinary stones can cause significant abdominal pain once such stones reach a size sufficient to impede urine flow through the ureter or urethra. Urinary stones may be formed from calcium, magnesium, ammonia, uric acid, cystine, and/or other compounds or combinations thereof.

Several methods can be used for treating patients with kidney stones, including observation, medical treatments (such as expulsion therapy), non-invasive treatments (such as extracorporeal shockwave lithotripsy (ESWL)), minimally-invasive or surgical treatments (such as ureteroscopy and percutaneous nephrolithotomy (“PCNL”)), and so on. In some approaches (e.g., ureteroscopy and PCNL), the physician gains access to the stone, the stone is broken into smaller pieces or fragments, and the relatively small stone fragments/particulates are extracted from the kidney using a basketing device and/or flushing (e.g., wash out, irrigation, aspiration). Flushing may occur peri-procedurally or post-procedural. For example, a therapy (e.g., alpha blockers) and/or a temporary urinary stent may be used to facilitate flushing the fragments from the target site.

In ureteroscopy procedures, a physician may insert a ureteroscope into the urinary tract through the urethra to remove urinary stones from the bladder and ureter. Typically, a ureteroscope includes an imaging device at its distal end configured to enable visualization of the urinary tract. The ureteroscope may also be paired with a lithotripsy device to capture or break apart urinary stones.

In PCNL procedures, which may be used to remove relatively large stones, a physician may insert a nephroscope through the skin (i.e., percutaneously) and intervening tissue to provide access to the treatment site for breaking-up and/or removing the stone(s). Fluidics can be applied to clear stone dust, small fragments, and/or thrombus from the treatment site and/or the visual field. In some instances, a relatively straight and/or rigid nephroscope is used, and the physician positions the tip of the nephroscope at the appropriate location within the kidney (e.g., calyx) by pushing/leveraging the device against the patient's body.

In other procedures, such as one or more of those discussed in further detail below, a physician can use multiple instruments via a percutaneous and/or direct access path to remove a kidney stone. For example, a physician can navigate a ureteroscope to a target site in a kidney through the urethra in a patient and insert a catheter device into the target site through the skin of the patient. The physician can use the ureteroscope and the catheter device in cooperation to fragment the kidney stone and extract the fragments from the patient. The ureteroscope may be used to locate and view the stone while the catheter device is used to fragment the stone and aspirate the fragments of the stone.

The present disclosure relates to systems, devices, and methods for navigating to and/or aspirating/irrigating a target site to perform a medical procedure. For example, a catheter can be used that includes an elongate shaft and a handle/base coupled to the shaft and configured to control actuation of the shaft (at least at a distal portion of the shaft). The shaft can include a lumen (e.g., working channel) configured to couple to an aspiration/irrigation system to provide aspiration/irrigation to a target site, such as to remove an object from a patient. The handle/base of the catheter can be controlled robotically and/or manually to articulate the distal portion of the shaft, so that the catheter can be navigated within the anatomy of a patient. For instance, the catheter can include multiple articulation elements (e.g., pull wires, cables, or other elongate movement members) that are coupled to the distal portion of the shaft and one or more manipulation components in the handle of the catheter. The articulation elements can be manipulated at the handle to control movement of the distal portion of the shaft. Additionally, or alternatively, the handle can be moved to control movement of the distal portion of the catheter, such as to insert/retract the tip of the catheter. A tip structure coupled to the distal end of the elongate shaft may include one or more shockwave elements configured to fragment the stone, and the fragments are removed from the patient by aspirating the stone fragments through the working channel of the elongate shaft. In some embodiments, the fragments may be removed post-operatively, such as the fragments being removed from the patient by urination. Additionally, the shockwave elements may also be used to adjust the position of the stone. As another example, a ureteroscope and a catheter may each include shockwave elements that help promote fragmentation of the stone. As another example, a ureteroscope can be used to locate and view the stone and a probe including one or more shockwave elements may be inserted through the catheter device to fragment the stone, the fragments of which may be aspirated via the ureteroscope and/or the catheter.

The techniques and devices discussed herein can enable objects to be removed from patients in an efficient manner that prevents damage to the anatomy of the patients and/or damage to the removal devices. For example, the articulable catheter structures discussed herein can enable a physician to navigate a distal portion of a catheter within a patient without moving an entirety of the catheter (e.g., by controlling one or more elements within a handle/base of the catheter).

In some implementations, the techniques discussed herein implement robotic-assisted medical procedures, wherein robotic tools enable a physician to perform endoscopic and/or percutaneous access and/or treatment for a target anatomical site. For example, the robotic tools can engage with and/or control one or more medical instruments, such as an endoscope (e.g., ureteroscope), catheter, probe, or another instrument, to access a target site in a patient and/or perform a treatment at the target site. In some cases, the robotic tools are guided/controlled by a physician. In other cases, the robotic tools operate in an automatic or semi-automatic manner. Although some techniques are discussed in the context of robotic-assisted medical procedures, the techniques may be applicable to other types of medical procedures, such as procedures that do not implement robotic tools or implement robotic tools for relatively few operations (e.g., less than a threshold number). For example, the techniques can be applicable to procedures in which a manually operated medical instrument is implemented, such as a manual catheter and/or scope controlled entirely by a physician.

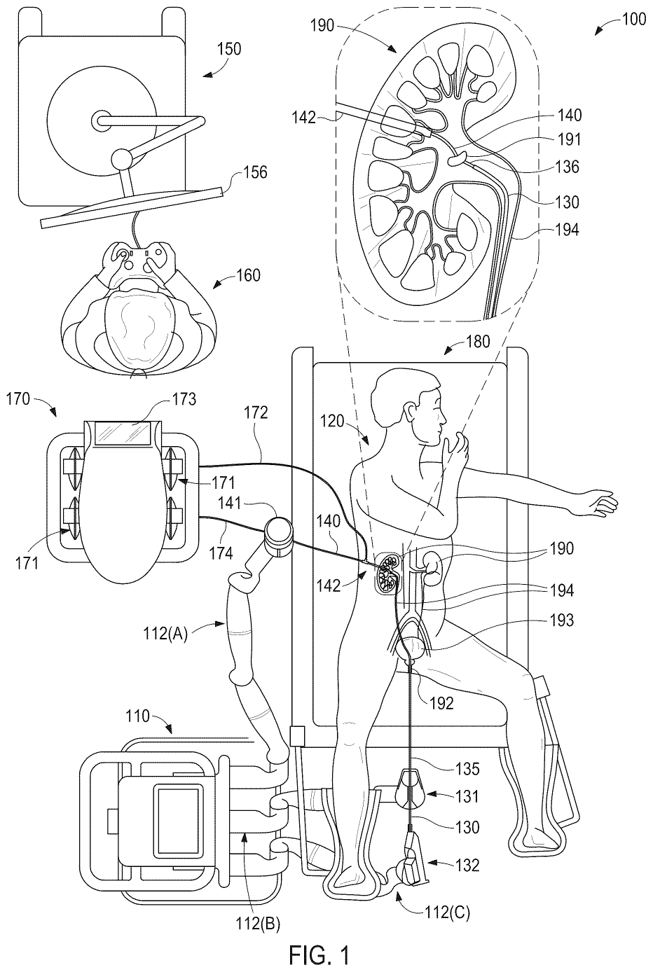

FIG. 1 illustrates an example robotic medical system 100 arranged for a diagnostic and/or therapeutic urology procedure in accordance with one or more embodiments. The medical system 100 includes a robotic system 110 configured to engage with and/or control one or more medical instruments to perform a procedure on a patient 120. In the example of FIG. 1, the robotic system 110 includes a scope 130 and a catheter 140, but could alternatively include other types of medical instruments.

The medical system 100 also includes a control system 150 configured to interface with the robotic system 110 and/or a physician 160, provide information regarding the procedure, and/or perform a variety of other operations. For example, the control system 150 can include a display 156 configured to present certain information to assist the physician 160 in performing the procedure.

The medical system 100 can also include a fluid management system 170 (sometimes referred to as “the aspiration system 170” or “the irrigation system 170”) configured to provide aspiration and/or irrigation to a target site, such as via the catheter 140, the scope 130, a percutaneous access device 142, and/or another medical device.

The medical system 100 can include a table 180 (e.g., bed) to hold the patient 120. Various acts are described herein as being performed by the physician 160. These acts can be performed directly by the physician 160, a user under the direction of the physician 160, another user (e.g., a technician), a combination thereof, and/or any other user.

The control system 150 can generally operate in cooperation with the robotic system 110 to perform the medical procedure. For example, the control system 150 can communicate with the robotic system 110 via a wired or wireless connection to control a medical instrument connected to the robotic system 110, receive images captured by a medical instrument, and so on. For example, the control system 150 can receive image data from the scope 130 (e.g., an imaging device associated with the scope 130) and display the image data (and/or representations generated therefrom) to assist the physician 160 in navigating the scope 130 and/or the catheter 140 within the patient 120. The physician 160 can provide input via an input/output (I/O) device, such as a controller, and the control system 150 can send control signals to the robotic system 110 to control movement of the scope 130 and/or catheter 140 connected to the robotic system 110. The scope 130 and catheter 140 (and/or another medical instrument) can be configured to move in a variety of manners, such as to articulate, roll, and so on.

In some embodiments, the control system 150 can provide power to the robotic system 110 via one or more electrical connections, provide optics to the robotic system 110 via one or more optical fibers or other components, and so on. In some embodiments, the control system 150 communicates with a medical instrument to receive sensor data (via the robotic system 110 and/or directly from the medical instrument). Sensor data can indicate or be used to determine a position and/or orientation of the medical instrument. In some embodiments, the control system 150 can communicate with an EM field generator (not illustrated) to control generation of an EM field around the patient 120.

Moreover, and in accordance with embodiments of the present disclosure, the control system 150 may be configured to communicate with shockwave elements arranged at the distal tip (or in any other location) of a catheter for the purpose of operating the shockwave elements to fragment (break apart) objects within the internal anatomy. In such embodiments, the control system 150 may also be in communication with (e.g., provide power to) or include a generator operable to provide the necessary electrical impulses to the shockwave elements for operation. The generator for the shockwave elements may be configured to generate voltage pulses to and through the shockwave elements (e.g., up to 10 kV and 60 Hz).

The robotic system 110 can include one or more robotic arms 112 configured to engage with and/or control one or more medical instruments. Each robotic arm 112 can include multiple arm segments coupled to joints, which can provide multiple degrees of movement. The distal end of each robotic arm 112 (e.g., end effector) can be configured to couple to a medical device. In the example of FIG. 1, the first robotic arm 112(A) is coupled to a handle 141 of the catheter 140. The second robotic arm 112(B) is coupled to a scope-driver instrument coupling/device 131, which can facilitate robotic control/advancement of the scope 130. Further, the third robotic arm 112(C) is coupled to a handle 132 of the scope 130, which can be configured to facilitate advancement and/or operation of a medical instrument deployed through the scope 130 (e.g., through a working channel of the scope 130). In this example, the second robotic arm 112(B) and/or the third robotic arm 112(C) can control movement of the scope 130 (e.g., articulation, roll, etc.). Although three robotic arms are connected to particular medical instruments in FIG. 1, the robotic system 110 can include any number of robotic arms that are configured to connect to any type of medical instrument.

The robotic system 110 can be communicatively coupled to any component of the medical system 100. For example, the robotic system 110 can be communicatively coupled to the control system 150 to receive control signals from the control system 150 to perform an operation, such as to control a robotic arm 112 in a particular manner, manipulate a medical instrument, and so on. Further, the robotic system 110 can be configured to receive an image (e.g., “image data”) from the scope 130 depicting internal anatomy of the patient 120 and/or send the image to the control system 150 to be displayed on the displays 156. Moreover, the robotic system 110 can be coupled to the control system 150 and/or the fluid management system 170 in a manner as to allow for fluids, optics, power, data, or the like to be received therefrom.

The fluid management system 170 is configured to provide/control aspiration and/or irrigation to a target site. The fluid management system 170 holds one or more fluid (i.e., IV) bags/containers 171 and/or controls fluid flow thereto/therefrom. For example, an irrigation line 172 may be fluidly coupled to the bags/containers 171 and to an irrigation port of a percutaneous access device 142. Irrigation fluid is provided to the target anatomy via the irrigation line 172 and the percutaneous access device 142. The fluid management system 170 may include certain electronic components, such as a display 173, flow control mechanics, and/or certain associated control circuitry. The fluid management cart 170 may comprise a stand-alone tower/cart that includes a pump with which aspiration fluid may be pulled into a collection container/cartridge via an aspiration channel/tube 174. The aspiration channel/tube 174 may be coupled to the catheter handle 141 to facilitate aspiration via a lumen in the catheter 140.

The percutaneous access device 142 is used to provide percutaneous access to a kidney 190 of the patient 120. The percutaneous access device 142 may include one or more sheaths and/or shafts through which instruments and/or fluids may access the target anatomy in which the distal end of the percutaneous access device 142 is disposed. In this example, the catheter 140 accesses the renal anatomy through the percutaneous access device 142. That is, the catheter 140 is inserted into the percutaneous access device 142 to access the target site.

Although various examples are discussed in the context of providing irrigation/aspiration via the catheter 140 and/or the percutaneous access device 142, irrigation fluid and/or aspiration may be provided to the treatment site (e.g., kidney 190) through another device, such as the scope 130.

A medical instrument (e.g., medical device) can include a variety of types of instruments, such as a scope (sometimes referred to as an “endoscope”), a catheter, a needle, a guidewire, a lithotripter, a basket retrieval device, forceps, a vacuum, a scalpel, an imaging probe, an imaging device, jaws, scissors, graspers, needle holder, micro dissector, staple applier, tacker, suction/irrigation tool, clip applier, and so on. A medical instrument can include a direct entry instrument, percutaneous entry instrument, and/or another type of instrument. In some embodiments, a medical instrument is a steerable device, while in other embodiments a medical instrument is a non-steerable device. In some embodiments, a surgical tool refers to a device that is configured to puncture or to be inserted through the human anatomy, such as a needle, a scalpel, a guidewire, and so on. However, a surgical tool can refer to other types of medical instruments.

The term “scope” or “endoscope” can refer to any type of elongate medical instrument having image generating, viewing, and/or capturing functionality (or configured to provide such functionality with an imaging device deployed though a working channel) and configured to be introduced into any type of organ, cavity, lumen, chamber, and/or space of a body. For example, a scope or endoscope, such as the scope 130, can refer to a ureteroscope (e.g., for accessing the urinary tract), a laparoscope, a nephroscope (e.g., for accessing the kidneys), a bronchoscope (e.g., for accessing an airway, such as the bronchus), a colonoscope (e.g., for accessing the colon), an arthroscope (e.g., for accessing a joint), a cystoscope (e.g., for accessing the bladder), a borescope, and so on. A scope/endoscope can comprise a rigid, flexible, or semi-flexible tube and/or may be dimensioned to be passed within an outer sheath, catheter, introducer, or other lumen-type device, or may be used without such devices. In some embodiments, a scope includes one or more working channels through which additional tools/medical instruments, such as lithotripters, basketing devices, forceps, laser devices, imaging devices, etc., can be introduced into a treatment site.

The terms “direct entry” or “direct access” can refer to any entry of instrumentation through a natural or artificial opening in a patient's body. For example, the scope 130 may be referred to as a direct access instrument, since the scope 130 enters into the urinary tract of a patient via the urethra.

The terms “percutaneous entry” or “percutaneous access” can refer to entry, such as by puncture and/or minor incision, of instrumentation through the skin of a patient and any other body layers necessary to reach a target anatomical location associated with a procedure (e.g., the calyx network of the kidney). As such, a percutaneous access device may refer to a medical instrument or assembly that is configured to puncture or to be inserted through skin and/or other tissue/anatomy, such as a needle, scalpel, guidewire, sheath, shaft, scope, catheter, and the like. However, it should be understood that a percutaneous access device can refer to other types of medical instruments in the context of the present disclosure. In some embodiments, a percutaneous access device refers to a device that is inserted or implemented with a device that facilitates a puncture and/or minor incision through the skin of a patient. For example, the catheter 140 may be referred to as a percutaneous access device when the catheter 140 is inserted through a sheath/shaft that is inserted into the skin of a patient.

In some embodiments, a medical instrument includes a sensor configured to generate sensor data. Sensor data can indicate a position and/or orientation of the medical instrument and/or can be used to determine a position and/or orientation of the medical instrument. For instance, sensor data can indicate a position and/or orientation of a scope, which can indicate roll orientation of a distal end of the scope. The position and orientation of a medical instrument can be referred to as the pose of the medical instrument. A sensor can be positioned on a distal end of a medical instrument and/or any other location. In some embodiments, a sensor can provide sensor data to the control system 150, the robotic system 110, and/or another system/device to perform one or more localization techniques to determine/track a position and/or an orientation of a medical instrument.

In some embodiments, a sensor can include an electromagnetic (EM) sensor with a coil of conductive material. Here, an EM field generator can provide an EM field that is detected by the EM sensor on the medical instrument. The magnetic field can induce small currents in coils of the EM sensor, which can be analyzed to determine a distance and/or angle/orientation between the EM sensor and the EM field generator. Further, a sensor can include another type of sensor, such as a camera, a range sensor (e.g., depth sensor), a radar device, a shape sensing fiber, an accelerometer, a gyroscope, an accelerometer, a satellite-based positioning sensor (e.g., a global positioning system (GPS)), a radio-frequency transceiver, and so on.

In some embodiments, the medical system 100 can also include an imaging device (not illustrated in FIG. 1) which can be integrated into a C-arm and/or configured to provide imaging during a procedure, such as for a fluoroscopy-type procedure. The imaging device can be configured to capture/generate one or more images of the patient 120 during a procedure, such as one or more x-ray or CT images. In examples, images from the imaging device can be provided in real-time to view anatomy and/or medical instruments within the patient 120 to assist the physician 160 in performing a procedure. The imaging device can be used to perform a fluoroscopy (e.g., with a contrast dye within the patient 120) or another type of imaging technique.

In some examples, the medical system 100 is implemented to perform a medical procedure relating to the renal anatomy, such as to treat kidney stones. For instance, robotic-assisted percutaneous procedures can be implemented, wherein robotic tools (e.g., one or more components of the medical system 100) can enable a physician/urologist to perform endoscopic (e.g., ureteroscopy) target access as well as percutaneous access/treatment. This disclosure, however, is not limited to kidney stone removal and/or robotic-assisted procedures. Although some embodiments of the present disclosure are presented in the context of catheters, nephroscopes, ureteroscopes, and/or the human renal anatomy, it should be understood that the principles disclosed herein may be implemented in any type of endoscopic/percutaneous procedure or another type of procedure.

In one illustrative and non-limiting procedure, the medical system 100 can be used to remove a kidney stone 191 from the patient 120. The physician 160 can position the robotic arms 112 of the robotic system 110 in the desired configuration and/or attach the appropriate medical instruments. For example, the physician 160 can position the first robotic arm 112(A) near a treatment site and attach an EM field generator (not illustrated), which can assist in tracking the location of the scope 130 and/or other instruments during the procedure. The physician 160 can then position the second robotic arm 112(B) between the legs of the patient 120 and attach the scope-driver instrument coupling 131, which can facilitate robotic control/advancement of the scope 130. In some instances, the physician 160 can insert a sheath 135 into the urethra 192 of the patient 120 and/or through the bladder 193 and up the ureter 194. The physician 160 can connect the sheath 135 to the scope-driver instrument coupling 131. The sheath 135 can include a lumen-type device configured to receive the scope 130, thereby assisting in inserting the scope 130 into the anatomy of the patient 120. However, in some embodiments the sheath 135 is not used (e.g., the scope 130 is inserted directly into the urethra 192). The physician 160 can then insert the scope 130 into the sheath 135 manually, robotically, or a combination thereof. The physician 160 can attach the handle 132 of the scope 130 to the third robotic arm 112(C), which can be configured to facilitate advancement and/or operation of a basketing device, laser device, and/or another medical instrument deployed through the scope 130.

The physician 160 interacts with the control system 150 to cause the robotic system 110 to advance and/or navigate the scope 130 into the kidney 190. The physician 160 navigates the scope 130 using a controller or other I/O device to locate the kidney stone 191. The control system 150 can provide information via the display 156 regarding the scope 130 to assist the physician 160 in navigating the scope 130, such as to view real-time images captured by the scope 130. In some embodiments, the control system 150 can use localization techniques to determine a position and/or an orientation of the scope 130, which can be viewed by the physician 160 through the display(s) 156. Further, other types of information can also be presented through the display(s) 156 to assist the physician 160 in controlling the scope 130, such as x-ray images of the internal anatomy of the patient 120.

Once at the site of the kidney stone 191 (e.g., within the calyx of the kidney 190), the scope 130 can be used to designate/tag a target location for the catheter 140 to access the kidney 190 percutaneously. To minimize damage to the kidney 190 and/or the surrounding anatomy, the physician 160 can designate a papilla as the target location for entering into the kidney 190 percutaneously. However, other target locations can be designated or determined. In some embodiments of designating the papilla, the physician 160 can navigate the scope 130 to contact the papilla, the control system 150 can use localization techniques to determine a location of the scope 130 (e.g., a location of the distal end of the scope 130), and the control system 150 can associate the location of the scope 130 with the target location. Further, in some embodiments, the physician 160 can navigate the scope 130 to be within a particular distance to the papilla (e.g., park in front of the papilla) and provide input indicating that the target location is within a field-of-view of the scope 130.

When the target location is designated, the catheter 140 can be inserted through a percutaneous access path into the patient 120 to reach the target site (e.g., rendezvous with the scope 130). For example, the catheter 140 can be connected to the first robotic arm 112(A) and the physician 160 can interact with the control system 150 to cause the robotic system 110 to advance and/or navigate the catheter 140. Alternatively, or additionally, the catheter 140 can be manually inserted and/or controlled, such as when the catheter 140 is implemented as a manually-controllable catheter. In some embodiments, a needle or another medical instrument is inserted into the patient 120 to create the percutaneous access path.

With the scope 130 and/or the catheter 140 located at the target location, the physician 160 can use the scope 130 and/or the catheter 140 to break up the kidney stone 191. The scope 130 and/or the catheter 140 can be used to extract pieces of the kidney stone 191 from the patient 120. The catheter 140 can provide aspiration to maintain/hold the kidney stone 191 at a distal end of the catheter 140 and/or at a relatively fixed position during the fragmentation procedure. The fluid management system 170 can provide irrigation to the target site via the percutaneous access device 142 and/or provide aspiration to the target site via the catheter 140 (e.g., a lumen in the catheter 140).

As one example, the physician 160 may use the catheter 140 to fragment the kidney stone 191. According to embodiments of the present disclosure, the catheter 140 may include one or more one or more shockwave elements at the distal end thereof and configured to generate shockwaves that fragment the kidney stone 191 into pieces. The pieces of the kidney stone 191 may be removed from the kidney 190 by aspirating the pieces through the percutaneous access path provided by the catheter 140. Additionally, some of the fragments may be removed by the scope 130, such as being aspirated through a working channel of the scope 130 or via a tool coupled to the scope 130, such as a basket.

As another example, the physician 160 may use both the scope 130 and catheter 140 to fragment the stone 191, such as fragmenting the kidney stone 191 from different sides as shown in FIG. 1. In some embodiments, the scope 130 and catheter 140 may be used to fragment the same side of the kidney stone 191. The catheter 140 can also suck out the pieces from the kidney 190 through the percutaneous access path. In some embodiments, the catheter 140 may include one or more shockwave elements to fragment the kidney stone 191, and a distal tip portion 136 of the scope 130 may also include one or more shockwave elements configured to generate shockwaves to fragment the kidney stone 191. In other embodiments, the catheter 140 may include one or more shockwave elements to fragment the kidney stone 191 while another a tool (e.g., a laser, a cutting instrument, lithotripter, etc.) may be deployed through the working channel of the scope 130 to fragment the kidney stone 191 into pieces. In other embodiments, the scope 130 may also include a laser filament that can be used to fragment the stone 191 while the catheter 140 includes one or more shockwave elements.

As another example, the physician may utilize the scope 130 having shockwave elements at the distal tip portion 136 to fragment the kidney stone 191 while the catheter 140, which does not include shockwave elements, is used to aspirate the fragments. The catheter 140 can provide aspiration to maintain/hold the kidney stone 191 at a distal end of the catheter 140 and/or at a relatively fixed position, while the shockwave elements of the scope 130 fragment the kidney stone 191.

Although various techniques/systems are discussed as being implemented as robotically-assisted procedures (e.g., procedures that at least partly use the medical system 100), the techniques/systems can be implemented in other procedures, such as in fully-robotic medical procedures, human-only procedures (e.g., free of robotic systems), and so on. For example, the medical system 100 can be used to perform a procedure without a physician holding/manipulating a medical instrument and without a physician controlling movement of a robotic system/arm (e.g., a fully-robotic procedure that relies on relatively little input to direct the procedure). That is, medical instruments that are used during a procedure can each be held/controlled by components of the medical system 100, such as the robotic arms 112 of the robotic system 110.

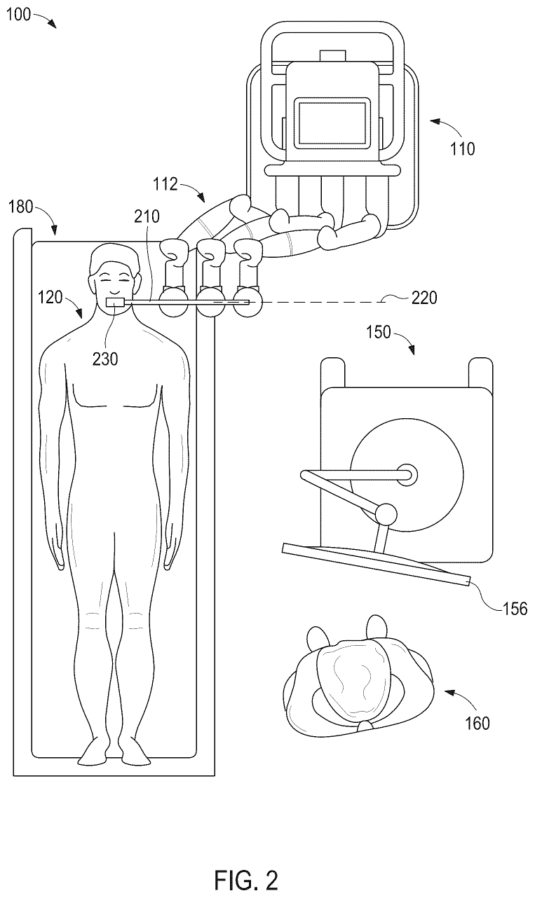

FIG. 2 illustrates the example robotic medical system 100 arranged for a diagnostic and/or therapeutic bronchoscopy procedure in accordance with one or more embodiments. The arm(s) 112 of the robotic system 110 may be configured to deliver a medical instrument, such as a steerable endoscope 210, which may be a procedure-specific bronchoscope for bronchoscopy, to a natural orifice access point (i.e., the mouth of the patient 120 positioned on the table 180 in the present example) to deliver diagnostic and/or therapeutic tools. The arrangement in FIG. 2 may also be utilized when performing a gastro-intestinal (GI) procedure with a gastroscope, a specialized endoscope for GI procedures.

Once the robotic system 110 is properly positioned, the robotic arms 112 may insert the steerable endoscope 210 into the patient robotically, manually, or a combination thereof. In some embodiments, the steerable endoscope 210 may comprise at least two telescoping parts, such as an inner leader portion and an outer sheath portion, with each portion coupled to a separate instrument driver from a set of instrument drivers and/or with each instrument driver coupled to the distal end of a respective robotic arm 112. This linear arrangement of the instrument drivers creates a “virtual rail” 220 that may be repositioned in space by manipulating the one or more robotic arms 112 into different angles and/or positions. Translation of one or more of the instrument drivers along the virtual rail 220 can advance or retract the endoscope 210 from the patient 120.

The endoscope 210 may be directed down the patient's trachea and lungs after insertion using precise commands from the robotic system 110 until reaching the target operative site. The use of separate instrument drivers can allow independent driving of separate portions of the endoscope 210. For example, the endoscope 210 may be directed to deliver a biopsy needle to a target, such as, for example, a lesion or nodule within the lungs of a patient. The needle may be deployed down a working channel that runs the length of the endoscope 210 to obtain a tissue sample to be analyzed by a pathologist. Depending on the pathology results, additional tools may be deployed down the working channel of the endoscope 210 for additional biopsies. For example, when a nodule is identified as being malignant, the endoscope 210 may endoscopically deliver tools to resect the potentially cancerous tissue.

In the arrangement of the system 100 in FIG. 2, a patient introducer 230 is attached to the patient 120 via a port (e.g., surgical tube). The patient introducer 230 may be secured to the table 180; e.g., via a patient introducer holder configured to support the introducer 230 and secure the position of the patient introducer 230 with respect to the table 180 or other structure. A curved tube component of the introducer 230 can connect the proximal and distal ends thereof and guide the instrument 210 through the introducer 230.

In some embodiments, one or more of the catheters discussed herein can be implemented in a bronchoscopy procedure, such as that illustrated in FIG. 2. For example, a catheter can be implemented in cooperation with or instead of the endoscope 210 to remove an object from the patient 120. In one illustration, a catheter and the endoscope 210 are interchanged on the robotic arms 112 and separately used to investigate/treat a target site. Here, the catheter can be inserted through the patient introducer 230 and used to provide aspiration/irrigation, such as to remove an object from the patient 120. In another illustration, a catheter is deployed through a working channel on the endoscope 210 to provide irrigation/aspiration. The catheter or the endoscope 210 may include one or more shockwave elements at the distal end thereof that can be used to promote the fragmentation of the object. The fragments may be removed by aspiration, such as being aspirated through the catheter.

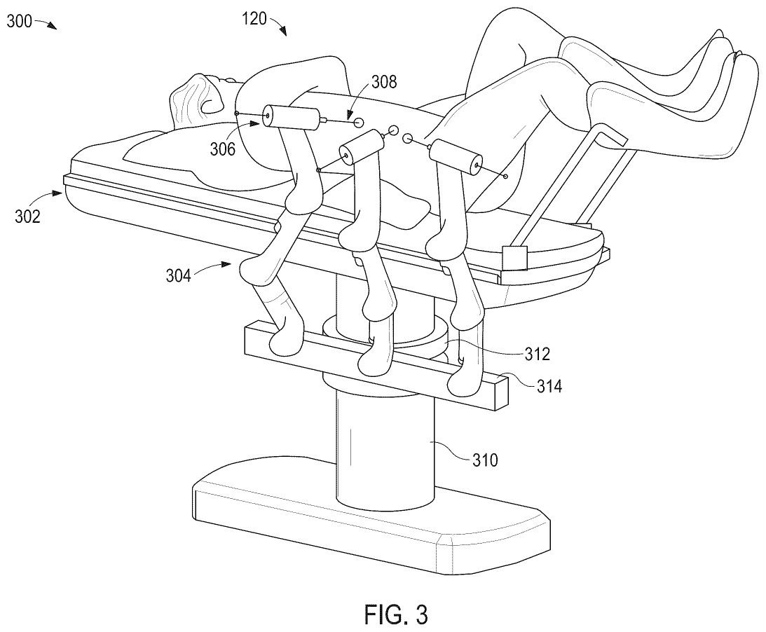

FIG. 3 illustrates a table-based robotic system 300 configured to perform a medical procedure in accordance with one or more embodiments. Here, one or more of the robotic components of the robotic medical system 100 can be incorporated into a table 302, which can reduce the amount of capital equipment within an operating room and/or allow greater access to the patient 120, in comparison to cart-based robotic systems. For example, the system 300 can include one or more components of the control system 150, the robotic system 110, and/or the fluid management system 170.

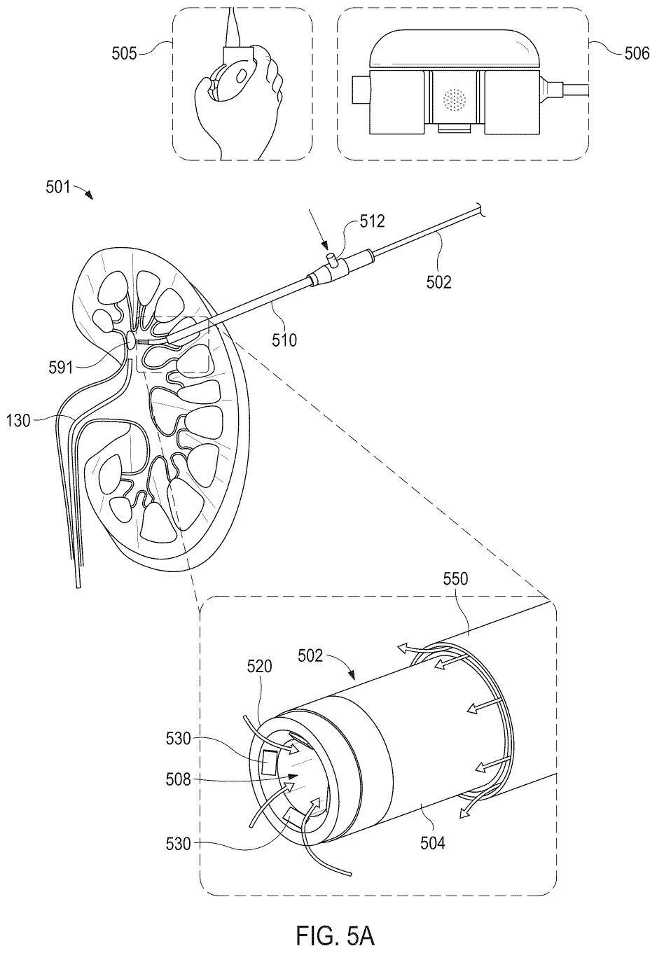

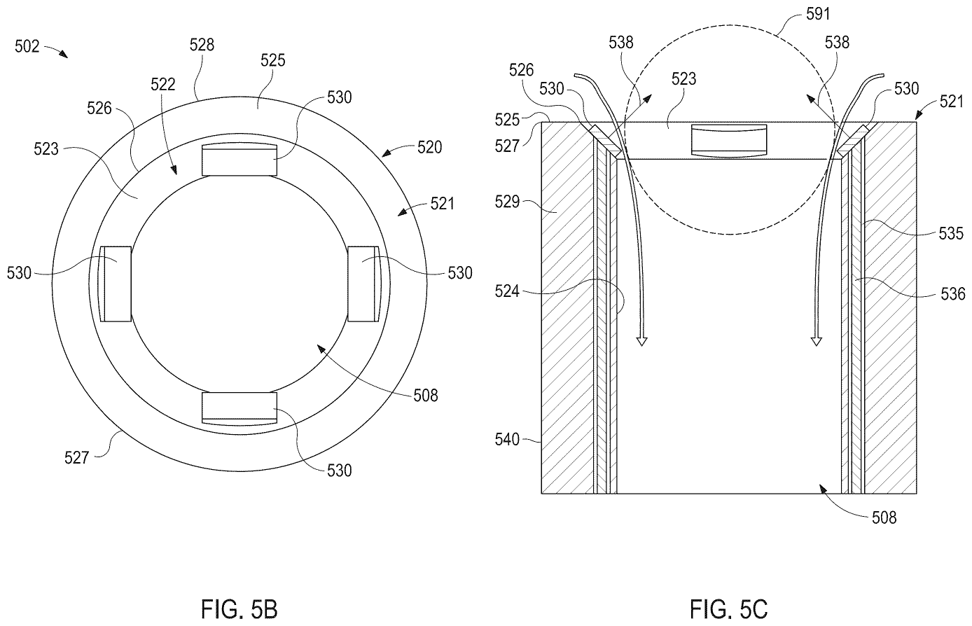

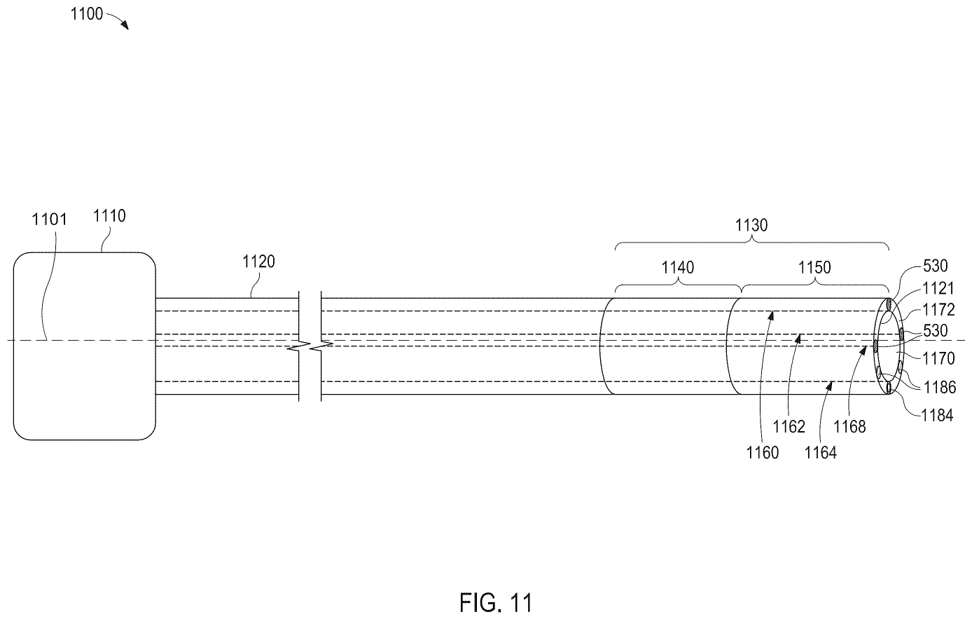

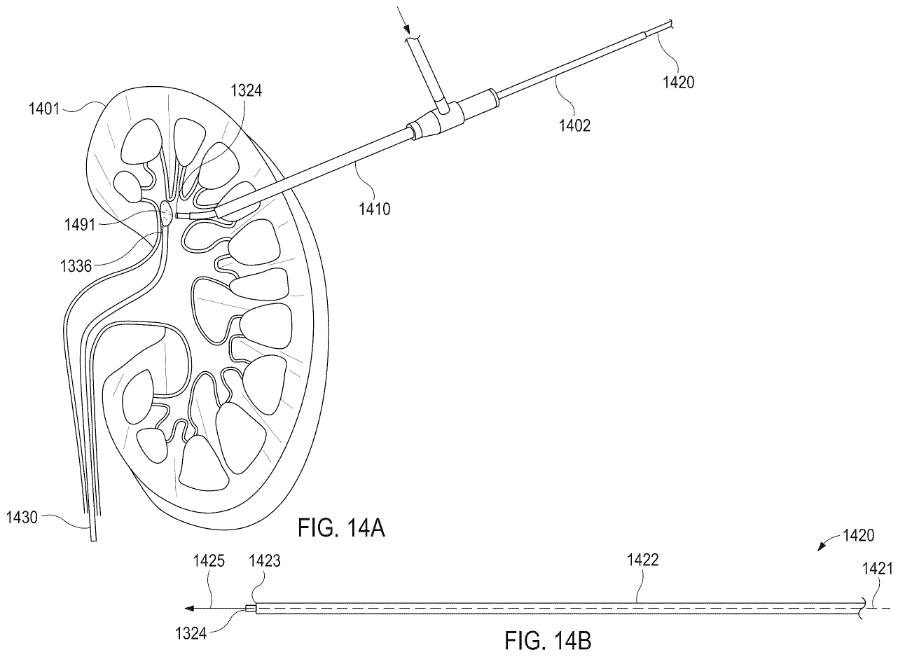

As shown, the table 302 can include one or more robotic arms 304 configured to engage with and/or control a medical device. Each robotic arm 304 can include multiple arm segments coupled to joints, which can provide multiple degrees of movement. A distal end of a robotic arm 304 (i.e., end effector 306) can be configured to couple to a device, which can include any of the medical devices discussed herein, such as a catheter (e.g., catheter 502 shown in FIG. 5A), scope (e.g., scope 1100 in FIG. 11), probe (e.g., probe 1420 shown in FIGS. 14A-14B), needle, etc. Each robotic arm 304 can be similar to or different than the robotic arms 112 of the system 100 of FIGS. 1 and 2. Further, each end effector 306 can be similar to or different than an end effector of the robotic system 110.

As shown, the robotic-enabled table system 300 can include a column 310 and one or more carriages 312 (e.g., ring-shaped movable structures) are mounted to the column 310, and from which the robotic arms 304 extend. The carriage(s) 312 translate along a vertical column interface that runs at least a portion of the length of the column 310 to provide different vantage points from which the robotic arms 304 may be positioned to reach the patient 120. The carriage(s) 312 may rotate around the column 310 to allow the robotic arms 304 to have access to multiples sides of the table 302. Rotation and/or translation of the carriage(s) 312 can allow the system 300 to align the medical instruments into different access points on the patient 120. The robotic arms 304 may be mounted on the carriage(s) 312 through one or more arm mounts 314, which may comprise a series of joints that may individually rotate and/or telescopically extend to provide additional configurability to the robotic arms 304. The column 310 may also convey power and control signals to the carriage(s) 312 and/or the robotic arms 304 mounted thereon.

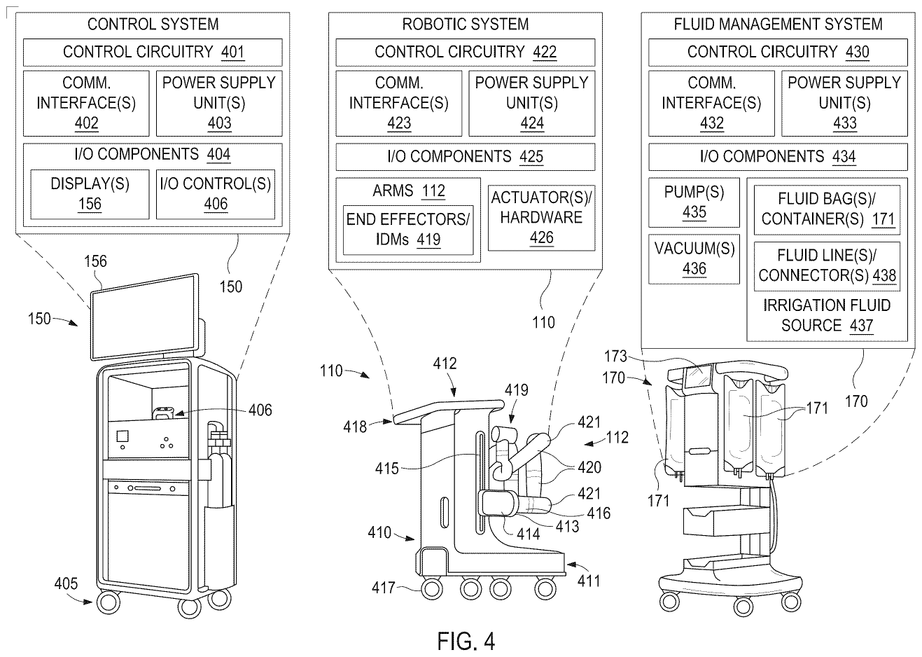

FIG. 4 illustrates medical system components that may be implemented in any of the medical systems of FIGS. 1-3 in accordance with one or more embodiments of the present disclosure. Although certain components are shown in FIG. 4, it should be understood that additional components not shown can be included in embodiments in accordance with the present disclosure. Furthermore, any of the illustrated components can be omitted, interchanged, and/or integrated into other devices/systems, such as the table 180, a medical instrument, etc.

The control system 150 can include one or more of the following components, devices, modules, and/or units (referred to herein as “components”), either separately/individually and/or in combination/collectively: control circuitry 401, one or more communication interfaces 402, one or more power supply units 403, one or more I/O components 404, and/or one or more mobilization components 405 (e.g., casters or other types of wheels). The control system 150 is illustrated as a cart-based system that is movable with the one or more mobilization components 405. After reaching the appropriate position, the mobilization components 405 can be immobilized using wheel locks to hold the control system 150 in place. The control system 150 can alternatively be implemented as a stationary system, integrated into another system/device, and so on.

The communication interfaces 402 can be configured to communicate with one or more devices/sensors/systems. For example, the communication interfaces 402 can send/receive data in a wireless and/or wired manner over a network.

The power supply units 403 can be configured to manage and/or provide power for the control system 150 (and/or the robotic system 110 and fluid management system 170, in some cases). The power supply units 403 include one or more batteries, such as a lithium-based battery, a lead-acid battery, an alkaline battery, and/or another type of battery. The power supply units 403 can comprise one or more devices and/or circuitry configured to provide a source of power and/or provide power management functionality.

The I/O components/devices 404 can include a variety of components to receive input and/or provide output, such as to interface with a user to assist in performing a medical procedure. The I/O components 404 can be configured to receive touch, speech, gesture, or any other type of input. In examples, the I/O components 404 can be used to provide input regarding control of a device/system, such as to control the robotic system 110, navigate a scope/catheter or other medical instrument attached to the robotic system 110 and/or deployed through the scope, control the table 180, control a fluoroscopy device, and so on. For example, a physician (not illustrated) can provide input via the I/O component(s) 404 and, in response, the control system 150 can send control signals to the robotic system 110 to manipulate a medical instrument. In examples, the physician can use the same I/O device to control multiple medical instruments (e.g., switch control between the instruments).

As shown, the I/O components 404 can include the one or more displays 156 configured to display data. The displays 156 include one or more touchscreens configured to receive input and/or display data. Further, the I/O components 404 can include one or more I/O devices/controls 406, which can include a touch pad, controller (e.g., hand-held controller, video-game-type controller, finger-based controls that enable finger-like movement, etc.), mouse, keyboard, wearable device (e.g., optical head-mounted display), virtual or augmented reality device (e.g., head-mounted display), foot panel (e.g., buttons at the user's feet), etc. Additionally, the I/O components 404 can include one or more speakers configured to output sounds based on audio signals and/or one or more microphones configured to receive sounds and generate audio signals.

In some embodiments, the I/O components 404 can output information related to a procedure. For example, the control system 150 can receive real-time images captured by a scope, such as the scope 130, and display the real-time images and/or visual/image representations of the real-time images via the display(s) 156. The display(s) 156 can present an interface(s), which can include image data from the scope and/or another medical instrument. Additionally, or alternatively, the control system 150 can receive signals (e.g., analog, digital, electrical, acoustic/sonic, pneumatic, tactile, hydraulic, etc.) from a medical monitor and/or a sensor associated with a patient, and the display(s) 156 can present information regarding the health or environment of the patient. Such information can include information that is displayed via a medical monitor including, for example, a heart rate (e.g., ECG, HRV, etc.), blood pressure/rate, muscle bio-signals (e.g., EMG), body temperature, blood oxygen saturation (e.g., SpO₂), CO₂, brainwaves (e.g., EEG), environmental and/or local or core body temperature, and so on.

The robotic system 110 can include an elongate support structure 410 (also referred to as a “column”), a robotic system base 411, and a console 412 at the top of the column 410. The column 410 can include one or more carriages 413 (also referred to as “the arm support 413”) for supporting deployment of the robotic arms 112. The carriage 413 can include individually configurable arm mounts that rotate along a perpendicular axis to adjust the base of the robotic arms 112 for positioning relative to a patient. The carriage 413 also includes a carriage interface 414 that allows the carriage 413 to vertically translate along the column 410. The carriage interface 414 can be connected to the column 410 through slots 415 positioned on opposite sides of the column 410 to guide the vertical translation of the carriage 413. The individually configurable arm mounts on the carriage 413 allow a robotic arm base 416 of the robotic arms 112 to be angled in a variety of configurations.

The base 411 can balance the weight of the column 410, the carriage 413, and/or robotic arms 112 over a surface, such as the floor. Accordingly, the base 411 can house heavier components, such as one or more electronics, motors, power supply, etc., as well as components that enable movement and/or immobilize the robotic system 110. The base 411 can include rollable wheels 417 (also referred to as “the casters 417” or “the mobilization components 417”) that allow for the robotic system 110 to move around the room for a procedure. The casters 417 can be immobilized using wheel locks to hold the robotic system 110 in place during the procedure. As shown, the robotic system 110 also includes a handle 418 to assist with maneuvering and/or stabilizing the robotic system 110. In this example, the robotic system 110 is illustrated as a movable cart-based system, but the robotic system 110 can alternatively be implemented as a stationary system, integrated into a table, and so on.

The robotic arms 112 include robotic arm bases 416 and end effectors 419, separated by a series of linkages 420 (also referred to as “arm segments 420”) that are connected by a series of joints 421. Each joint 421 can comprise an independent actuator and each actuator can comprise an independently controllable motor. Each independently controllable joint 421 represents an independent degree of freedom available to the robotic arm 112. For example, each of the arms 112 can have seven joints, and thus provide seven degrees of freedom. However, any number of joints can be implemented with any degrees of freedom.

The end effector 419 of each robotic arm 112 can comprise an instrument device manipulator (IDM). In some embodiments, the IDM can be removed and replaced with a different type of IDM. For example, a first type of IDM can manipulate an endoscope, a second type of IDM can manipulate a catheter, a third type of IDM can hold an EM field generator, and so on. In some instances, an IDM can include connectors to transfer pneumatic pressure, electrical power, electrical signals, and/or optical signals to/from the robotic arm 112. The IDMs may be configured to manipulate medical instruments using techniques including, for example, direct drives, harmonic drives, geared drives, belts/pulleys, magnetic drives, and the like. In some embodiments, the IDMs can be attached to respective ones of the robotic arms 112, wherein the robotic arms 112 are configured to insert or retract the respective coupled medical instruments into or out of the treatment site.

The robotic arms 112 are configured to control a position, orientation, and/or articulation of a medical instrument (e.g., a sheath and/or a leader of a scope) attached thereto. For example, the robotic arms 112 can be configured/configurable to manipulate a scope or catheter using articulation elements. The articulation elements can include one or more pull wires, cables, fibers, and/or flexible shafts. To illustrate, the robotic arms 112 can be configured to actuate multiple pull wires of the scope/catheter to deflect the tip of the scope/catheter. Pull wires can include any suitable or desirable materials, such as metallic and/or non-metallic materials such as stainless steel, Kevlar, tungsten, carbon fiber, and the like. In some embodiments, the scope/catheter is configured to exhibit nonlinear behavior in response to forces applied by the articulation elements. The nonlinear behavior can be based on stiffness and/or compressibility of the scope/catheter, as well as variability in slack or stiffness between different articulation elements.

The console 412 is positioned at the upper end of column 410 of the robotic system 110. The console 412 can include a display(s) to provide a user interface for receiving user input and/or providing output (e.g., a dual-purpose device, such as a touchscreen), such as to provide a physician/user with pre-operative data, intra-operative data, information to configure the robotic system 110, and so on.

The robotic system 110 can also include control circuitry 422, one or more communication interfaces 423, one or more power supply units 424, one or more input/output components 425, and one or more actuators/hardware 426. The communication interfaces 423 can be configured to communicate with one or more device/sensors/systems. For example, the communication interfaces 423 can send/receive data in a wireless and/or wired manner over a network.

The power supply units 424 can be configured to manage and/or provide power for the robotic system 110. The one or more power supply units 424 can include one or more batteries, such as a lithium-based battery, a lead-acid battery, an alkaline battery, and/or another type of battery. The power supply units 424 can comprise one or more devices and/or circuitry configured to provide a source of power and/or provide power management functionality. In some applications, the power supply units 424 include a connector that is configured to couple to the control system 150 to receive power from the control system 150.

The I/O components/devices 425 can be configured to receive input and/or provide output, such as to interface with a user. The I/O components 425 can be configured to receive touch, speech, gesture, or any other type of input. In examples, the I/O components 425 can be used to provide input regarding control of a device/system, such as to control/configure the robotic system 110. The I/O components 425 can include the one or more displays configured to display data.

The actuators/hardware 426 can be configured to facilitate movement of the robotic arms 112. Each actuator 426 can comprise a motor, which can be implemented in a joint or elsewhere within a robotic arm 112 to facilitate movement of the joint and/or a connected arm segment/linkage. In some embodiments, a user can manually manipulate a robotic arm 112 without using electronic user controls. For example, during setup in a surgical operating room or at any point during a procedure, a user may select a button on a distal end of a robotic arm 112 to enable an admittance control mode and then manually move the robotic arm 112 to a particular orientation/position.

The robotic fluid management system 170 can include control circuitry 430, one or more communication interfaces 432, one or more power supply units 433, one or more input/output components 434, one or more pumps 435, one or more vacuums 436, and an irrigation fluid source 437. The communication interfaces 432 can be configured to communicate with one or more device/sensors/systems. For example, the communication interfaces 432 can send/receive data in a wireless and/or wired manner over a network.

The power supply units 433 can be configured to manage and/or provide power for the fluid management system 170. The power supply units 433 include one or more batteries, such as a lithium-based battery, a lead-acid battery, an alkaline battery, and/or another type of battery. The power supply units 433 can comprise one or more devices and/or circuitry configured to provide a source of power and/or provide power management functionality. The power supply units 433 include a connector configured to couple to the control system 150 to receive power from the control system 150.

The fluid management system 170 can be configured to control the pump(s) 435 and/or the vacuum(s) 436 to provide irrigation/aspiration. For example, a medical instrument may be attached to the pump(s) 435/vacuum 436 to provide irrigation/aspiration to a target site via medical instrument. In examples, the fluid management system 170 can include one or more flow meters, valve controls, and/or other fluid-/flow-control components (e.g., sensor devices, such as pressure sensors) in order to provide controlled irrigation and/or aspiration/suction capabilities for a medical instrument. In some embodiments, the control system 150 and/or the robotic system 110 can generate and provide one or more signals to the fluid management system 170 to control irrigation/aspiration.

The pump(s) 435 can be attached to an irrigation fluid source 437, which can include the fluid bag(s)/container(s) 171 and/or a fluid line(s)/connector(s) 438 to connect to a medical instrument(s). The pump(s) 435 can pump irrigation fluid (e.g., saline solution) through one or more medical instruments and into a treatment site. In some examples, the pump(s) 435 is a peristaltic pump(s). In some embodiments, the pump(s) 435 can be replaced with a vacuum that is configured to apply a vacuum pressure to draw the irrigation fluid from the irrigation fluid source 437 and out through the respective coupled medical instrument. Although FIG. 4 includes the pump(s) 435, in some embodiments, irrigation fluid flow is achieved without the use of pumps, wherein such flow is driven primarily by gravitational force.

The vacuum(s) 436 can be configured to facilitate fluid aspiration. For example, the vacuum(s) 436 can be configured to apply a negative pressure to draw fluid out of a treatment site. The vacuum(s) 436 may be connected to a collection container into which withdrawn fluid is collected. In some examples, aspiration suction may be facilitated by one or more pumps rather than a vacuum.

FIG. 5A illustrates an example catheter 502 and a percutaneous access device 510 disposed at least partly in a kidney 501 of a patient in accordance with one or more embodiments. The catheter 502 and percutaneous access device 510 may be representative any of the catheters and percutaneous access devices discussed herein. In this example, the instruments 502, 510 are illustrated in the context of a urology procedure to treat/remove a kidney stone 591 from the kidney 501. However, the instruments 502, 510 can be used in other types of procedures. As noted above, urology procedures and/or other types of procedures can be implemented manually at least in part and/or can be performed using robotic technologies at least in part.

The catheter 502 can be articulated relative to a distal end/tip of the catheter 502 in a variety of directions. In examples, the catheter 502 can be configured to move with two degrees of freedom (2-DOF) (e.g., two of x, y, z, yaw, pitch, or roll movement). To illustrate, the distal end portion of the catheter 502 can be configured to move right/left or up/down (e.g., x, y, or z movement) and also move to insert/retract the catheter 502 (e.g., translate along the x, y, or z axis). In other examples, the catheter 502 can be configured to move with 3-DOF (e.g., three of x, y, z, yaw, pitch, or roll movement). To illustrate, the distal end portion of the catheter 502 can be configured to move right/left and up/down (e.g., two of x, y, or z movement) and also move to insert/retract the catheter 502. However, the catheter 502 can also be configured to move with 4-DOF (e.g., x, y, z, and pitch/yaw/roll movement), 6-DOF (e.g., x, y, z, pitch, yaw, and roll movement), and so on. In some embodiments, such as when the catheter 502 is implemented with a robotically-controllable handle, the catheter 502 is not configured for roll movements. However, the catheter 502 can be configured for roll and/or other types of movement in some cases, such as when the catheter 502 is configured with a manually-controllable handle or some robotically-controllable cases.

The catheter 502 can be implemented with the percutaneous access device 510 to provide aspiration and/or irrigation to the kidney 501. The percutaneous access device 510 may include one or more sheaths and/or shafts through which instruments (e.g., the catheter 502) and/or fluids may access the target anatomy in which the distal end of the device 510 is disposed. In some embodiments, active aspiration (e.g., suction) may be drawn through a lumen 508 (e.g., working channel) of the catheter 502 to a proximal end of the catheter 502 (e.g., a handle of the catheter 502). In some embodiments, irrigation can be provided via the percutaneous access device 510, such as between concentric sheaths. For example, a fluid management system (not illustrated) can be connected to an irrigation port 512 to provide irrigation to the percutaneous access device 510, which travels down the percutaneous access device 510 to the target site. FIG. 5A illustrates an example of the flow of aspiration fluid into the lumen 508 of the catheter 502 and the flow of irrigation fluid from the percutaneous access device 510. In some embodiments, a passive aspiration outflow channel may be formed in the space between the outer wall of the catheter 502 and an inner wall/sheath of the percutaneous access device 510. When the catheter 502 is disposed within the percutaneous access device 510, the catheter 502 and the shaft(s)/sheath(s) of the percutaneous access device 510 may be generally concentric. The catheter 502 and the percutaneous access device 510 may have generally circular cross-sectional shapes over at least portions thereof.

The catheter 502 may be controllable in any suitable or desirable way, either based on manual control and/or robotic control. In FIG. 5A, handles/bases 505, 506 provide examples that may be used to control the catheter 502. The first handle 505 illustrates a hand-held/manual handle configured to be manipulated by a physician/user to control movement of the catheter 502. Meanwhile, the second handle 506 illustrates a robotically controllable handle manipulated by a robotic arm (e.g., an end effector of a robotic arm) to control movement of the catheter 502. By implementing an articulable catheter, the techniques/structures can allow various positions within the patient to be reached in a manner that prevents/minimizes damage to the anatomy of the patient. For example, a physician can navigate the distal portion of the catheter 502 to reach a particular cavity in the kidney 501 (e.g., calyx) where a kidney stone is located, without repositioning the rest of a shaft 504 of the catheter 502 and/or the percutaneous access device 510.

In some embodiments, the catheter 502 is free of an imaging device/camera used to capture image data of an internal anatomy of the patient. However, in other embodiments the catheter 502 can include an imaging device(s), such as on the tip of the catheter 502. Further, in embodiments, the catheter 502 is implemented without a position sensor (i.e., does not include a position sensor). However, the catheter 502 can be implemented with a position sensor in some cases, such as on a distal end of the catheter 502.

In some embodiments, the scope 130 is inserted into the kidney 501 through the urethra, bladder, and ureter. The scope 130 may assist in accurately positioning the catheter 502, such as including an imaging device to help visualize the catheter 502 until it is located in a desired position relative to the kidney stone 591. Additionally, the scope 130 may be used to move the kidney stone 591 into position relative to the distal end of the catheter 502, such as moving the kidney stone 591 to the calyx that the catheter 502 has entered.

The distal end of the catheter 502 includes a tip structure 520 (e.g., filter feature, containment structure) coupled to the shaft 504 that is configured to engage with an object (e.g., kidney stone) at a target site within a patient. According to embodiments of the present disclosure, the tip structure 520 may include a one or more shockwave elements 530 (e.g., shockwave emitters) arranged at or near the opening to the lumen 508 and configured to generate shockwaves to promote fragmentation of objects (e.g., the kidney stone 591). The tip structure 520 may also be configured to inhibit objects or fragments that are larger than a particular size (diameter) from being sucked into the shaft 504 of the catheter 502 and potentially forming a clog within the lumen 508.

In the context of a urological procedure, the tip structure 520 can be positioned at a target site and used to fragment one or more kidney stones 591 and aspirate kidney stone fragments from the kidney 501. In some embodiments, the tip structure 520 is configured to hold the kidney stone 591 during fragmentation, as shown in FIG. 5C, such as by being held in place by the fluid aspirating through the lumen 508, while the shockwave elements 530 are activated to fragment (break apart) the kidney stone 591. In some embodiments, the kidney stone 591 may also be fragmented simultaneously by an instrument deployed from another device at the target site, such as being fragmented by the scope 130 which is shown as a ureteroscope in FIG. 5A. Although the tip structure 520 can be implemented as a separate component from the rest of the shaft 504, the tip structure 520 can be integral with rest of the shaft 504 or implemented in other manners.

In instances where the tip structure 520 is implemented as a separate component from the rest of the shaft 504, the tip structure 520 can be attached to the shaft 504 with an adhesive, a fastener, an interlocking mechanism (e.g., tabs, grooves, etc.), and so on. Additionally, while the shaft 504 and the tip structure 520 are shown as having a substantially cylindrical form in FIG. 5A, the shaft 504 and/or tip structure 520 may assume other forms, such as a polygonal shape (e.g., triangular rectangular, square, hexagonal, octagonal, etc.).

The tip structure 520 can be made of a material that avoids degradation in certain contexts, such as catastrophic degradation. For example, the tip structure 520 can be made of stainless steel (or other types of steel), titanium, tungsten, and/or other materials that may have relatively high melting points above a threshold and/or that can generally maintain its structure when laser beams emanating from a scope, such as a ureteroscope 130, and/or shockwaves produced by shockwave elements 530 inadvertently and/or occasionally contact the tip structure 520. However, the tip structure 520 and/or any other portion of the shaft 504 can be implemented with other materials.

FIGS. 5B and 5C illustrate end and cross-sectional views, respectively, of the tip structure 520 according to one or more embodiments of the present disclosure. The lumen 508 terminates at a distal end 521 of the tip structure 520. The distal end 521 includes an end surface 525 (e.g., end face) with an outer periphery 527 (e.g., outer edge) and an inner periphery 526 (e.g., inner edge) that defines the entrance to the lumen 508. In some embodiments, the end surface 525 may comprise an annular end surface that circumscribes and helps define the lumen 508. In some embodiments, the end surface 525 may be a planar surface (e.g., planar end face).

The tip structure 520 has inner surface 522 partially defining the lumen 508. In some embodiments, and as shown in FIG. 5B, the inner surface 522 has a first inner surface portion 523 and a second inner surface portion 524. The first inner surface portion 523 is closer to the distal end 521 than the second inner surface portion 524. The first inner surface portion 523 is a tapered (angled) surface that extends from the inner periphery 526 to the second inner surface portion 524. The lumen 508 may have a constant or substantially constant inner diameter defined by the second inner surface portion 524, but the diameter of the first inner surface portion 523 changes (varies) due to the tapered surface. The tapered first inner surface portion 523 is shown as a generally conical surface. The tip structure 520 also includes an outer surface 540 opposite the inner surface 522. The outer surface 540 is shown as a generally cylindrical surface that extends to the outer periphery 527 of the end surface 525. The outer surface 540, however, can be any suitable shape depending on the shape of the tip structure 520.

In some embodiments, the shockwave elements 530 are positioned on the first surface portion 523. While four shockwave elements 530 are shown in FIG. 5B and arranged with equidistant spacing along the first inner surface portion 523, any number of shockwave elements 530 with equal or unequal spacing may be arranged on the first inner surface portion 523. The arrangement may be based on desired impact of the shockwaves, safety, efficacy, and range. In some embodiments, the shockwave elements 530 may alternatively (or in addition thereto) be positioned on the end surface 525. Additionally, one or more shockwave elements 530 may be arranged on the second inner surface portion 524 in addition to one or more shockwave elements 530 being located at the first inner surface portion 523 or at the end surface 525. Locating the shockwave elements 530 at the second inner surface portion 524 may, for example, be selected in order to facilitate reducing the size of the fragments that flow through, or material that clogs, the remainder of the lumen 508 and also mitigates the formation of clogs within the portion of the lumen 508 defined by the second inner surface portion 524.

The object, such as a kidney stone 591, may be at least partially received within the portion of the lumen 508 defined by the inner surface 522 to position the stone 591 adjacent the shockwave elements 530. The first inner surface portion 523 is tapered to allow at least a portion of the object to be received within the tip structure 520. In other words, the first inner surface portion 523 may define a cup sized to at least partially receive the object within. The second inner surface portion 524 may have an inner diameter selected to inhibit the object and large fragments thereof from passing through the remainder of the catheter 502 to avoid developing obstructions in the lumen 508. Fluid may be aspirated through the lumen 508 while the object is at least partially disposed within the tip structure 520, such as when the shockwave elements 530 are generating shockwaves.

In at least one embodiment, operation of the shockwave elements 530 and the aspiration functionality can be specifically timed to coincide. In such embodiments, the control system 150 (FIGS. 1 and 4) may communicate with a generator that operates the shockwave elements 530 and the fluid management system 170 (FIGS. 1 and 4). Consequently, the two features may be configured to operate simultaneously and in concert with each other.

In some embodiments, one or more of the shockwave elements 530 may be in direct contact with the kidney stone 591 during the fragmentation process. In other embodiments, the shockwave elements 530 are in proximity to the kidney stone 591 but are not in direct contact therewith, such as being separated by a gap. The catheter 502 may be maneuvered to place the shockwave elements 530 in a desired proximity of the kidney stone 591, such as at a desired gap distance, to facilitate fragmentation of the kidney stone 591.

FIG. 5C illustrates a schematic cross-sectional view of the tip structure 520 with a kidney stone 591 (shown by dashed lines) partially received within the tip structure 520. As shown, the tapered surface of the first inner surface portion 523 allows the kidney stone 591 to partially enter the tip structure 520 without passing fully into the lumen 508 during aspiration. Arranging the shockwave elements 530 on the first inner surface portion 523 positions the shockwave elements 530 around the kidney stone 591, which helps promote fragmentation of the stone 591 during operation. In some embodiments, the kidney stone 591 may be engaged with (e.g., contacting, captured at, etc.) the first inner surface portion 523 while the shockwave elements 530 are activated (operated) to produce shockwaves.

The shockwave elements 530 shown in FIGS. 5A-5C are shown schematically as rectangular elements, but the shape is not limited to polygonal geometries. In operation, the shockwave elements 530 form a bubble in the fluid within the patient that collapses. Collapsing the bubble results in the formation of a shockwave (e.g., acoustic wave) that promotes fragmentation of the object. Each shockwave element 530 may comprise an electrode pair that are energized to create the shockwaves. The electrode pair includes first and second electrodes separated by a gap, and shockwaves can be generated at the shockwave element 530 by applying a high voltage pulse across the first and second electrodes. For example, the pulses may be between 1 kV to 10 kV, such as 3 kV. Each pulse initially ionizes the conductive fluid proximate to the electrodes, and at some point a plasma arc forms across the gap between the electrode pairs, creating a low impedance path where current flows freely. Thermal energy from the plasma arc heats the conductive fluid creating a rapidly expanding vapor bubble. The expansion and subsequent collapse of the vapor bubble creates an acoustic shockwave that generates (propagates) through the conductive fluid. In some embodiments, the shockwave elements 530 are laser elements that use a laser beam to heat up the conductive fluid proximate to the shockwave element 530 to similarly create a vapor bubble to create acoustic waves.

In some embodiments, the shockwave elements 530 comprise piezoelectric elements that change shape in response to an electric current to generate (propagate) shockwaves toward the object. For example, the shockwave element 530 may include a metallic element, such as a diaphragm, which vibrates in response to an applied current to generate (propagate) acoustic waves.

Each shockwave element 530 may receive energy via a connection 536 that extends through the catheter 502 to the shockwave element 530. When the shockwave elements 530 are either the electrode pairs or the piezoelectric element, then the connection 536 may comprise one or more wires that electrically connect the electrode pairs or the piezoelectric element to a power source. When the shockwave element 530 is a laser element, the connection 536 may comprise a fiber optic filament that guides a laser beam that exits the shockwave element 530, which may be the end of the fiberoptic filament.

In some embodiments, the tip structure 520 includes one or more internal lumens 535 formed in a wall 529 of the catheter 502 that provide access for each connection 536, such as one or more wires extending through the catheter 502. In embodiments where the shockwave element 530 is an electrode pair, the connection 536 may comprise a pair of wires, each wire extending to a separate electrode of the electrode pair through the same or different internal lumen 535. Housing the wires in separate internal lumens 535 helps avoid shorts between the wires within the catheter 502. In other embodiments, the shockwave elements 530 could be wired in series, without departing from the scope of the disclosure.

In some applications, the shockwaves produced by the shockwave elements 530 generally propagate at a trajectory (e.g., direction) perpendicular to the surface where located, as represented by arrows 538. In other applications, the shockwaves will propagate 180 degrees from the point of generation, unless a reflective element (usually metallic) is used to direct the generated shockwave. In the illustrated application, the metallic structure of the electrodes and the tip structure 520 will act as a reflective element, thereby resulting in shockwaves that propagate in a hemisphere around the arrow 538. Accordingly, the arrows 538 represent the propagation direction of the shockwaves emitted from the shockwave elements 530 toward the object 591. The angle of the first inner surface portion 523 may be selected based on the desired trajectory of the shockwaves, with the first inner surface portion 523 being angled to face toward the center of the tip structure 520. The first inner surface portion 523 is shown being tapered at about 45 degrees. However, the first inner surface portion 523 may be tapered at an angle more or less than 45 degrees, such as about 60 degrees or about 30 degrees. In some embodiments, the angle of the first inner surface portion 523 and arrangement of the shockwave elements 530 may be selected such that the shockwaves converge to a common focal point. In some embodiments, the inner surface 522 does not have a tapered portion (e.g., the first inner surface portion 523) and instead has a uniform inner diameter that the shockwave elements 530 are arranged around.

In some embodiments, the shockwave elements 530 are activated simultaneously to create shockwaves. In other embodiments, each of the shockwave elements 530 may be activated independent of the other shockwave elements 530. For example, the shockwave elements 530 may be activated in series to facilitate compounding the shockwaves produced by the shockwave elements 530 to fragment the object, such as the kidney stone 591.

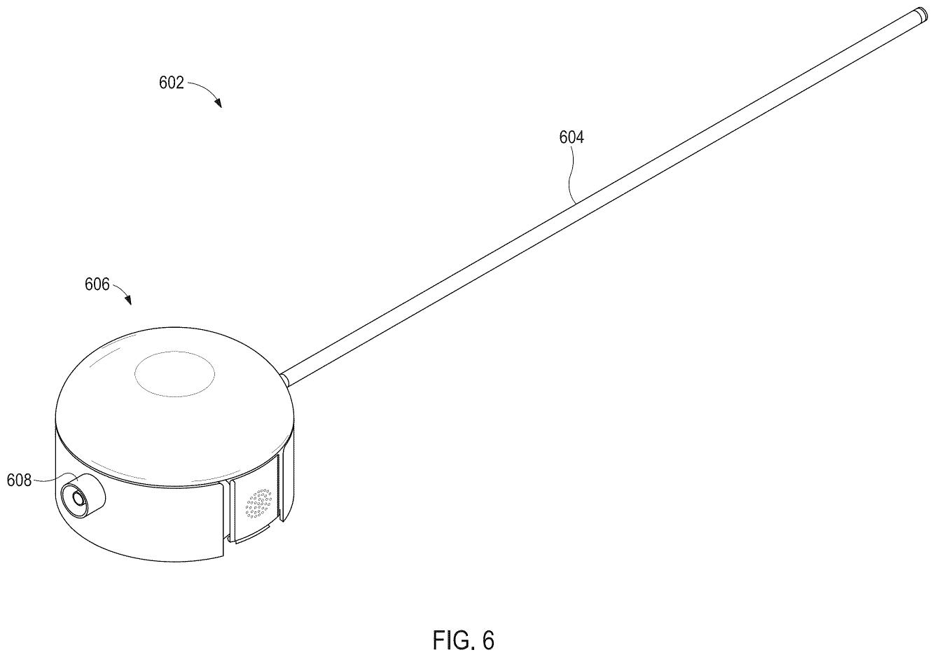

FIG. 6 illustrates example features of a controllable catheter 602 that may be used in accordance with one or more embodiments of the present disclosure. The features of the catheter 602 may be implemented in the context of one or more of the catheters discussed herein. As illustrated, the catheter 602 includes an elongate shaft 604 connected to a base or “handle” 606 configured to control actuation of at least a portion of the elongate shaft 604. The elongate shaft 604 extends through and is otherwise fluidly coupled to a port 608 provided by the handle 606, and the port 608 can be connected to a fluid management system and/or another system to facilitate aspiration, irrigation, deployment of an instrument through a working channel of the catheter 602, and so on.

At least a portion of the shaft 604 can be formed of various materials, such as plastics, rubbers, vertebrae links, metal or plastic braids/coils, and so on, such that at least a portion of the shaft 604 is flexible for articulation. In some embodiments, the shaft 604 includes reinforcement material (e.g., braided) to strengthen and/or facilitate flexibility of the shaft 604. For example, the shaft 604 can include braid reinforcement for hoop strength and or to prevent kinking of the shaft 604 when the shaft 604 is navigated within the anatomy of a patient. Further, in some embodiments, the shaft 604 includes multiple layers of material that are implemented in a variety of configurations to facilitate the features of the shaft 604 discussed herein.

FIG. 6 shows the handle 606 as a robotically controllable handle that can be coupled to a robotic arm and/or another device interface (e.g., sterile adapter). While the base 606 is shown as being robotically controllable, the instrument base 606 may alternatively be manually controllable, such as the handle 505 shown in FIG. 5A. Although certain handles are discussed in the context of being implemented in a manually-controllable catheter or robotically-controllable catheter, the disclosed catheters can be implemented in other contexts. For example, a manually-controllable catheter can include robotic components to be implemented as a robotically-controllable catheter (e.g., secondary use as a robotic catheter), and/or a robotically-controllable catheter can include manual component to be implemented as a manually-controllable catheter (e.g., secondary use as a manual catheter). As such, in some cases, a catheter is configured for both manual and robotic manipulation.

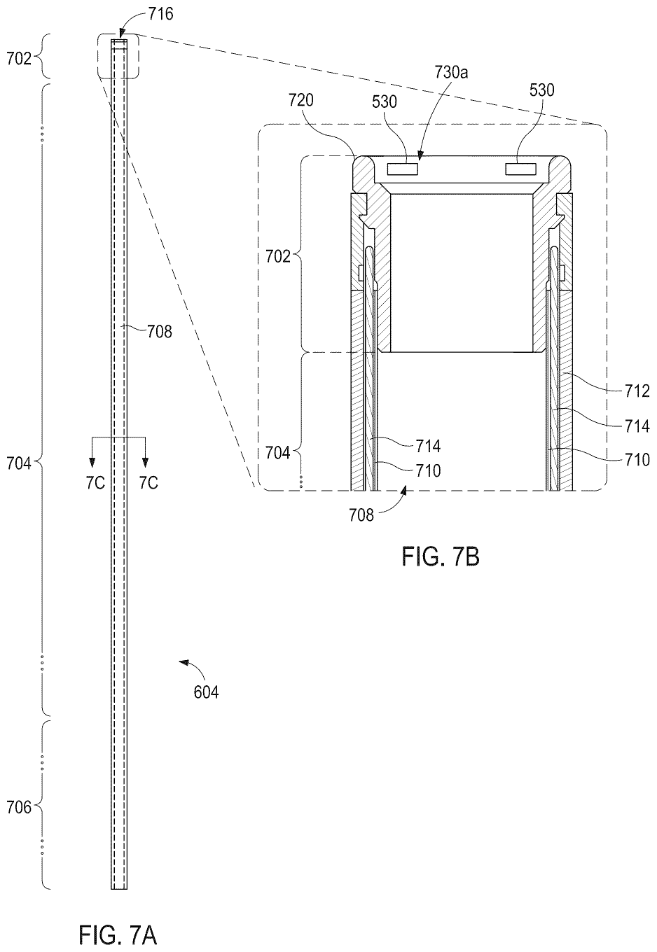

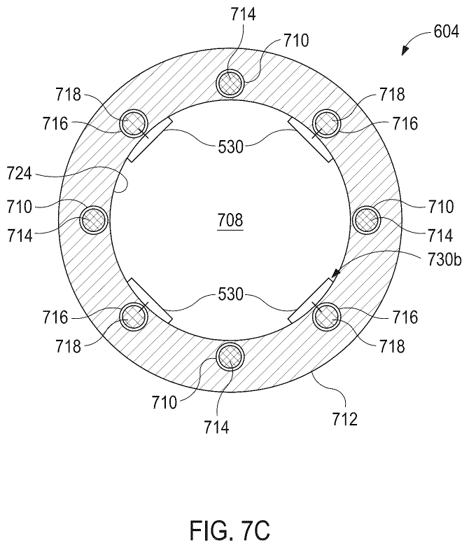

FIGS. 7A-7C depict enlarged views of the shaft 604 and its component parts, according to one or more embodiments. As illustrated, the shaft 604 can include a distal tip portion 702 (e.g., distal section, distal portion), a middle portion 704 (e.g., medial portion), a proximal portion 706 (e.g., proximal section), and a lumen 708 that extends through at least a portion of the shaft 604. For example, the lumen 708 can extend through the entirety of the shaft 604 from the distal tip portion 702 to the proximal section 706, which may be connected to the port 608 of the handle 606 (FIG. 6). However, the lumen 708 can extend other distances through the catheter 602. In examples, the lumen 708 can be referred to as a working channel. The distal tip portion 702, the middle section 704, and/or the proximal section 706 can each exhibit any longitudinal length.

The terms distal, middle/medial, proximal, and/or other terms are used to describe a position of a feature relative to another feature. For example, a proximal feature of the catheter 602 can refer to a feature that is farthest from a target or anatomical site (e.g., during use/a procedure), whereas a distal feature of the catheter 602 can refer to a feature that is closest to the target or anatomical site.

In some embodiments, as best seen in FIG. 7B, the distal tip portion 702 includes a tip structure 720 that includes one or more shockwave elements 530. The tip structure 720 may be similar in some respects to the tip structure 520 of FIGS. 5A-5C. While the tip structure 720 is shown as a separate component from the rest of the shaft 604, in other embodiments the tip structure 720 can be integral with rest of the shaft 604 or implemented in other manners. In some cases, the tip structure 720 is formed of a different material than the rest of the shaft 604.

As shown in FIGS. 7B-7C, the shaft 604 of the catheter 602 can include one or more first lumens 710 defined in a wall 712 of the shaft 604, such as an outer wall. The first lumens 710 can be spaced equidistantly apart around the wall of the shaft 604 or at another location. The catheter 602 includes one or more articulation elements 714 slidably disposed in the one or more first lumens 710. The articulation elements 714 may comprise pull wires, cables, fibers, and/or flexible shafts, and can be made of any suitable or desirable material, such as metallic and non-metallic materials, including stainless steel, Kevlar, tungsten, carbon fiber, and the like. The catheter 602 can exhibit nonlinear behavior in response to forces applied by the articulation elements 714. The nonlinear behavior may be based on stiffness and/or compressibility of the catheter 602, as well as variability in slack or stiffness between different articulation elements 714. Although a particular number of first lumens 710 and articulation elements 714 are illustrated in the figures, any number of lumens and/or articulation elements can be implemented.

The articulation elements 714 can be attached/extend to the distal tip portion 702 of the shaft 604. At a proximal end, the articulation elements 714 can be coupled to corresponding components housed within the handle 606 (FIG. 6) (e.g., an input assembly) and operable to control articulation of the shaft 604, such as by deflecting the distal tip portion 702 of the shaft 604. The articulation elements 714 can be manipulated to change the pose of the catheter 602 (FIG. 6) and to selectively change the position the tip structure 720 at the target site, such as positioning the tip structure 720 adjacent to an object at the target site. The handle 606 can be operated to pull (and/or release tension of) the articulation elements 714 within corresponding first lumens 710 to cause the distal tip portion 702 to deflect from a longitudinal axis. In some embodiments, the catheter 602 is configured to move in two directions based on manipulation of the one or more articulation elements 714 (e.g., up/down or right/left). In other embodiments, the catheter 602 is configured to move in four directions based on manipulation of the one or more articulation elements 714 (e.g., up/down and right/left). In yet other embodiments, the catheter 602 is configured to move in other directions. In some robotic examples, the catheter 602 can move in any direction by using a combination of four primary directions and four articulation elements.

FIG. 7C is an enlarged, cross-sectional view of the shaft 604 taken along section line 7C-7C in FIG. 7A. As shown, one or more second lumens 716 may be defined in the wall 712 to accommodate connections 718 (e.g., lead wires) configured to connect the shockwave elements 530 to a power source or laser source. Each connection 718 is disposed in a corresponding one of the second lumens 716 and extends to a respective shockwave element 530 of the tip structure 720. The second lumens 716 may be the same as or otherwise aligned with lumens 535 of FIG. 5C, which are formed in the wall of the tip structure 720.

In some embodiments, as shown in FIG. 7B, the catheter 602 includes a first set of shockwave elements 730a arranged at the tip structure 720. As shown in FIG. 7C, the catheter 602 may further include a second set of shockwave elements 730b arranged within the shaft 604 about the lumen 708. The first set of shockwave elements 730a includes one or more shockwave elements 530 that are positioned to fragment an object (e.g., a kidney stone) adjacent the tip structure 720. The second set of shockwave elements 730b include one or more additional shockwave elements 530 positioned within the elongate shaft 604 downstream of the tip structure 720 and arranged on an inner surface 724 that defines the lumen 708. The second set of shockwave elements 730b may be configured to help break-up fragments of an object flowing through the lumen 708 and thereby help prevent (avoid) clogs. The second set of shockwave elements 730b may be arranged in the middle section 704, but could alternatively be arranged in the distal or proximal sections 702, 706, or a combination of any of the foregoing, without departing from the scope of the disclosure.

To inhibit clogs from forming in the lumen 708, the second set of shockwave elements 730b may be placed along the shaft 604 at a location where the shaft 604 tends to bend as the pose is changed. For example, the second set of shockwave elements 730b may be placed before, after, and/or along an articulating region (e.g., bendable section) of the catheter 602. While the second set of shockwave elements 730b is shown in FIG. 7C with four shockwave elements 530 spaced equidistantly around the lumen 708, the second set of shockwave elements 730b can have any number and/or spacing around the lumen 708. For example, the second set of shockwave elements 730b can have one, two, three, four, five, six, seven, or more shockwave element 530.

In some embodiments, the second set of shockwave elements 730b may include a plurality of shockwave elements 530 arranged at the same axial position along the lumen 708. Alternatively, the second set of shockwave elements 730b may include shockwave elements 530 that are axially spaced from each other along the length of the lumen 708. In some embodiments, the catheter 602 may include a plurality of second sets of shockwave elements 730b incorporated into the shaft 604, with each set being located at a different axial positions (spaced) along the length of the lumen 708. For example, the middle section 704 and/or the proximal section 706 may include one or more second sets of shockwave elements 730b. In some embodiments, the distal tip portion 702 may also include one or more sets of shockwave elements 530 downstream of the tip structure 720.

In some embodiments, and as shown in FIG. 7C, each shockwave element 530 in the second set of shockwave elements 730b may be connected to a power source or laser source via a connection extending through corresponding second lumens 716. In some embodiments, one or more shockwave elements 530 in the first set of shockwave elements 730a and one or more shockwave elements 530 in the second set of shockwave elements 730b may share a common second lumen 716. For example, the connection 718 in the second lumen 716 may include one or more lead wires or filaments that lead to multiple shockwave elements 530 in different sets 730a, b. Thus, the connection 718 may be configured to allow for independent control of different shockwave elements 530 that share the same connection 718. In other embodiments, shockwave elements 530 that share a common connection 718 are activated simultaneously rather than being independently activated. In some embodiments, the first and second sets of shockwave elements 730a, b have separate connections 536 (FIG. 5C) that are housed in separate lumens formed in the wall 712, such as third lumens (not show) formed in the wall 712 that extend to the second set of shockwave elements 730b.

The second set of shockwave elements 730b may be operated by activating the corresponding shockwave elements 530 independently or simultaneously. In some embodiments, the second set of shockwave elements 730b may be operated by activating the shockwave elements 530 in series to facilitate compounding the shockwaves produced by the shockwave elements 530 to break-up the fragments flowing through the lumen 708.

While FIG. 7C depicts four first lumens 710 with a corresponding articulation element 714 arranged within each first lumen 710, and four second lumens 716 with a corresponding connection 718 (e.g., lead wire) arranged within each second lumen 710, it is contemplated herein to include to first lumens 710 with a corresponding articulation element 714 arranged within each first lumen 710, and two second lumens 716 with a corresponding connection 718 (e.g., lead wire) arranged within each second lumen 710. In such embodiments, the first and second lumens 710, 716 may be angularly spaced from each other equidistantly or non-equidistantly, without departing from the scope of the disclosure. Moreover, in such embodiments, shaft rule may be facilitated at the handle.

As shown, one or more second lumens 716 may be defined in the wall 712 to accommodate connections 718 (e.g., wires) configured to connect the shockwave elements 530 to a power source or laser source. Each connection 718 is disposed in a corresponding one of the second lumens 716 and extends to a respective shockwave element 530 of the tip structure 720. The second lumens 716 may be the same as or otherwise aligned with lumens 535 of FIG. 5C, which are formed in the wall of the tip structure 720.

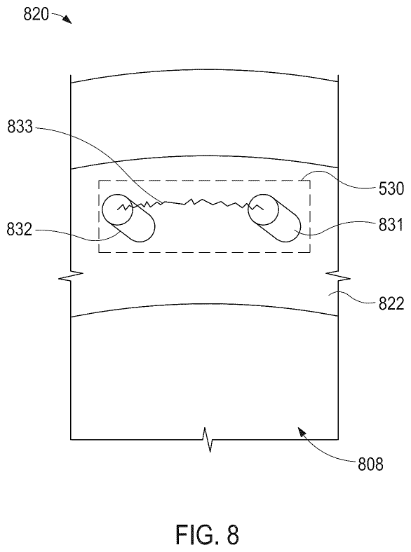

FIG. 8 is a schematic perspective view of a portion of an example tip structure 820 that can be incorporated into a catheter described herein. One shockwave elements 530 is shown as including a first electrode 831 and a second electrode 832 protruding from an inner surface 822 of the tip structure 820 that partially defines the lumen 808. The first and second electrodes 831, 832 are separated from one another by a gap. Shockwaves can be generated at the shockwave element 530 by applying a high voltage pulse, such as pulses between 1 kV to 10 kV, across the first and second electrodes 831, 832. Each pulse initially ionizes the conductive fluid proximate to the first and second electrodes 831, 832. At some point, a plasma arc 833 forms across the gap between the first and second electrodes 831, 832, creating a low impedance path where current flows freely. Thermal energy from the plasma arc heats the conductive fluid creating a rapidly expanding vapor bubble. The expansion and subsequent collapse of the vapor bubble creates an acoustic shockwave that propagates through the conductive fluid.

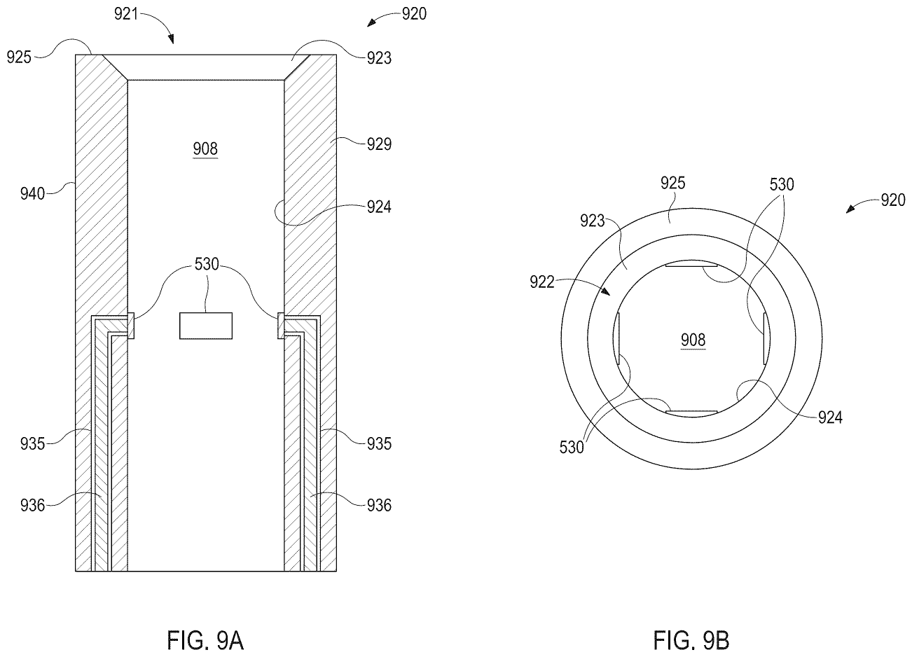

FIGS. 9A-9B illustrate an example tip structure 920 that includes a set of one or more shockwave elements 530, according to one or more additional embodiments. The tip structure 920 may be incorporated into one of the catheters described herein, such as being incorporated into a catheter that is being used in conjunction with another surgical device configured to fragment an object, such as a kidney stone, within the patient. For example, the tip structure may be partially inserted into the elongated shaft 604 similar to tip structure 720 shown in FIG. 7B. The tip structure 920 includes the set of shockwave elements 530 to break-up fragments being aspirated into the catheter and thereby help to inhibit the formation of clogs. In the context of a urological procedure, a ureteroscope may be used to fragment a kidney stone while a catheter with the tip structure 920 is used to aspirate the fragments through the lumen 908.

FIG. 9A illustrates a cross-sectional view of the tip structure 920, while FIG. 9B illustrates an end view of the tip structure 920. The tip structure 920 has an end surface 925 at the distal end 921 that surrounds (circumscribes) the entrance to a lumen 908. The tip structure 920 has inner surface 922 partially defining the lumen 908 and an outer surface 940 opposite the inner surface 922 and defining the exterior of the tip structure 920. In some embodiments, the inner surface 922 has a first inner surface portion 923 and a second inner surface portion 924. The first inner surface portion 923 is a tapered surface that extends from the end surface 925 to the second inner surface portion 924.

The lumen 908 may have a constant or substantially constant inner diameter defined by the second inner surface portion 924 while the inner diameter of the lumen 908 varies along the length of the first inner surface portion 923 due to the tapered surface. The shockwave elements 530 are arranged about the second inner surface portion 924 and operable to break up fragments flowing through the portion of the lumen 908 defined by the inner surface 922. While four shockwave elements 530 are shown arranged with equidistant angular spacing from each other, any number of shockwave elements 530 with equal or unequal angular spacing may be included. The tip structure 920 may also include lumens 935 formed in a wall 929 thereof that provide a pathway for a connection 936 (e.g., a wire) that connects a respective shockwave element 530 to a power source or a laser source.

In some instances, the object (e.g., a kidney stone) in the target site may be located in a position that is difficult to reach with an articulatable (steerable) catheter, such as being in a location that cannot be accessed with the catheter without causing undesirable damage to the patient's anatomy, such as being in a “dead zone” that cannot be reached due to the articulation limits of the catheter. For example, the object may be located behind the tip structure of the catheter, which prevents the object from being received within the tip structure due to limits on articulation of the catheter. The object may need to be dislodged from its position or broken up into smaller fragments so that object (or pieces thereof) can be received into the tip structure, such as being drawn to the tip structure by aspiration through the lumen (e.g., working channel) of the catheter. To be able to properly locate and break up the object, the tip structure may have a first set of shockwave elements that includes first and second arrays of shockwave elements 530. The first array of shockwave elements 530 is positioned to generate shockwaves in multiple directions that can be used to dislodge and/or fragment an object that is located in a position that inhibits receiving the object in the tip structure. The second array of shockwave elements 530 is located to fragment the dislodged object or a large fragment of the object interfacing with the front of the tip structure to facilitate aspiration of object fragments. The tip structure may also include a second set of shockwave elements positioned to inhibit the formation of clogs in the lumen of the catheter, such as being positioned downstream of the tip structure within the shaft.

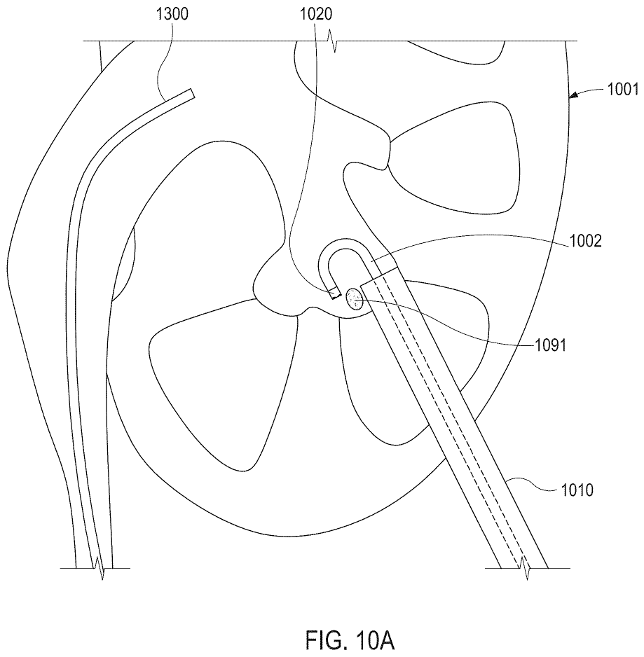

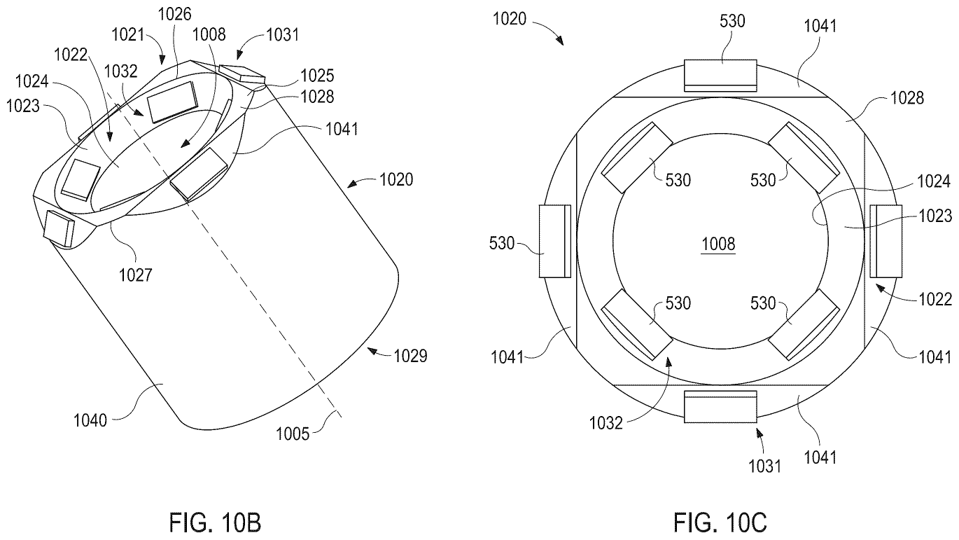

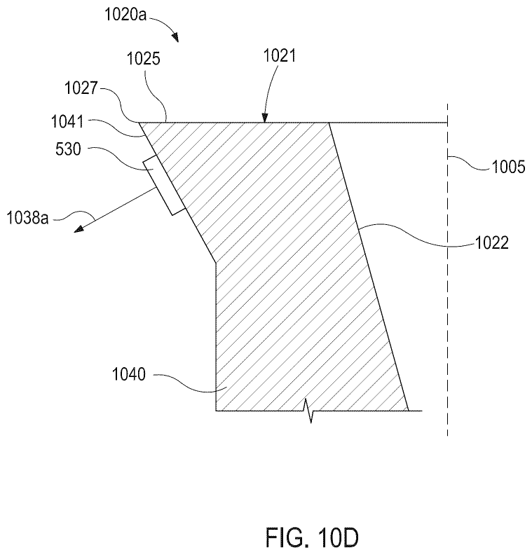

FIG. 10A is a schematic of a target site that includes a kidney stone 1091 currently inaccessible to a catheter 1002 advanced into a kidney 1001 through a percutaneous access device 1010. The kidney stone 1091 may be located in a feature (e.g., a calyx) of the kidney 1001, and attempts to use a ureteroscope 1300 to dislodge and/or move the kidney stone 1091 into a more desirable position for the catheter 1002 may have failed. The kidney stone 1091 is also in a location that inhibits engaging fragmentating with an array of shockwave elements 530 on an inner side of the tip structure 1020, such as being located within a “dead zone” that is not readily reachable due to the articulation limits of the catheter 1002 as shown in FIG. 10A.

The catheter 1002 can be used to generate (propagate) shockwaves that may dislodge the kidney stone 1091, or create fragments that can be engaged with the tip structure 1020 to facilitate aspiration and/or additional fragmentation. The kidney stone 1091 may be dislodged due to the impact of the shockwaves or due to turbulence in the surrounding environment caused by the shockwaves. More specifically, the catheter 1002 may be articulated to place the tip structure 1020 adjacent to the kidney stone 1091 and operated to generate (propagate) shockwaves with a first array of shockwave elements 1031 (shown in FIGS. 10B-10C) in an attempt to dislodge and/or break-up the kidney stone 1091. The dislodged kidney stone 1091, or the resulting fragments thereof, can thereafter be engaged with the tip structure 1020 such that a second array of shockwave elements 1032 (shown in FIGS. 10B-10C) can break up the kidney stone 1091 to facilitate aspiration of the resulting fragments. In some embodiments, the ureteroscope 130 is used to maneuver the dislodged kidney stone 1091, or large fragments thereof, into position with respect to the second array of shockwave elements 1032.

FIGS. 10B and 10C illustrate perspective and end views, respectively, of the tip structure 1020, according to one or more embodiments of the disclosure. As illustrated, the tip structure 1020 has inner surface 1022 defining a portion of a lumen 1008 that extends through the catheter 1002. A distal end 1021 of the tip structure 1020 may include an end surface 1025 having an inner periphery 1026 (e.g., inner edge) that defines the entrance of the lumen 1008. The end surface 1025 may include a plurality of contiguous planar portions 1028 that circumscribe the lumen 1008. In some embodiments, the end surface 1025 may include one or more contiguous portions or one or more discontinuous portions. The tip structure 1020 also includes an outer surface 1040 opposite of the inner surface 1022. In some embodiments, the first array of shockwave elements 1031 are arranged on the outer surface 1040 while the second array of shockwave elements 1032 are arranged on the inner surface 1022.

The inner surface 1022 has a first inner surface portion 1023 and a second inner surface portion 1024. The first inner surface portion 1023 may comprise a tapered surface that extends from the inner periphery 1026 to the second inner surface portion 1024. The portion of the lumen 1008 within the tip structure 1020 may have a constant or substantially constant inner diameter defined by the second inner surface portion 1024 while the inner diameter of the lumen 1008 varies along the of the first inner surface portion 1023 due to the tapered surface. The tapered first inner surface portion 1023 is shown as a generally conical surface that extends inward away from the end surface 1025 at an inward facing angle, shown as about 45 degrees relative to the longitudinal axis 1005 in FIG. 10B. In other words, the tapered first inner surface portion 1023 is angled to face inwards towards the lumen 1008 (e.g., face towards a longitudinal axis of the tip structure 1020).

The outer surface 1040 is shown as being a generally cylindrical surface with a plurality of tapered outer surfaces 1041 around the periphery of distal end 1021 that extend away from an outer periphery 1027 of the end surface 1025 at an outward facing angle. In other words, the plurality of outward facing surfaces are angled to face outwards away from the lumen 1008. Thus, the tapered outer surfaces 1041 are swept back surfaces relative to the distal end 1021 and angled away from a longitudinal axis 1005 of the tip structure 1020, while the tapered first inner surface portion 1023 is angled to face towards the longitudinal axis 1005. The outer surface 1040 is shown with four tapered outer surfaces 1041 at the distal end 1021 that are separated by planar portions 1028 of the end surface 1025. The outer surface 1040 may include any number of tapered outer surfaces 1041, such as one, two, three, four, five, six, or more tapered outer surfaces 1041. Additionally, the tapered outer surfaces 1041 may be equally or unequally spaced around the periphery of the distal end 1021 of the tip structure 1020.

The first array of shockwave elements 1031 is shown including four shockwave elements 530 that are each located on separate tapered outer surfaces 1041 and arranged with equidistant spacing around the circumference of the tip structure 1020. The number of shockwave elements 530 of the first array of shockwave elements 1031 may be equivalent to or less than the number of tapered outer surfaces 1041. For example, each tapered outer surface 1041 may have at least one shockwave element 530 arranged thereon.

The arrangement and outward angle of the tapered outer surfaces 1041 are selected to facilitate placing the first array of shockwave elements 1031 in position to advantageously interact with the kidney stone 1091. For example, the four tapered outer surfaces 1041 are arranged so that the shockwave elements 530 are angularly offset from each other by about 90 degrees (e.g., located at clock positions of 3 o'clock, 6 o'clock, 9 o'clock, and 12 o'clock). The catheter 1002 can be manipulated to rotate the tip structure 1020 about the longitudinal axis 1005 to place at least one of the shockwave elements 530 of the first array of shockwave elements 1031 in position to interact with the kidney stone 1091. The outward angle is oriented such that the shockwave element 530 coupled thereto emits shockwaves in a desired trajectory (e.g., direction). The shockwaves produced by the shockwave elements 530 propagate at a trajectory that is generally perpendicular to the tapered outer surface and otherwise in a hemispherical shape extending from the tapered outer surface. The tapered outer surfaces 1041 are shown as being generally planar surfaces with an outward facing angle of about 45° relative to the longitudinal axis 1005. The tapered outer surfaces 1041 are angled to direct the shockwaves produced by the shockwave elements 530 in a forward trajectory that is at an angle relative to the longitudinal axis 1005. This allows the tip structure 1020 to interact with a kidney stone 1091 that cannot be readily reached by the catheter 1002 to interface the kidney stone 1091 with the second array of shockwave elements 1032.

In some embodiments, the tapered outer surface 1041 may be oriented at any outward angle between 0° and 90° relative to the distal end 1021 as schematically represented by the outward angle shown in FIG. 10B. For example, when oriented at 0°, the tapered outer surface 1041 is coplanar with the distal end 1021 and the shockwaves are directed directly forward of the distal end 1021 at a trajectory generally parallel to the longitudinal axis 1005. When oriented at 90°, the tapered outer surfaces 1041 extend parallel to the longitudinal axis 1005 such that the shockwaves are propagated away from the outer surface 1040 generally perpendicular to the longitudinal axis 1005. When oriented between 0° and 90° (e.g., excluding 0° and 90°), the shockwaves are propagated in a generally forward trajectory relative to the distal end 1021.

The first inner surface portion 1023 may similarly be oriented at the inward facing angle between 0° and 90°. For example, when oriented at 0°, the first inner surface portion 1023 is coplanar with the distal end 1021 and the shockwaves are directed directly forward of the distal end 1021 at a trajectory generally parallel to the longitudinal axis 1005. When oriented at 90°, the first inner surface portion 1023 extends parallel to the longitudinal axis 1005 and shockwaves are propagated generally perpendicular to the longitudinal axis 1005, similar to the shockwave elements 530 of FIGS. 9A-9B. When oriented between 0° and 90° (e.g., excluding 0° and 90°), the shockwaves are propagated in a generally forward trajectory to break up an object engaged with the tip structure 1020.

In other embodiments, the tip structure 1020 may be used in applications where shockwaves need to be propagated in a trajectory that is generally backwards (proximally) from the distal end 1021. For example, the kidney stone 1091 may be located at a severe angle behind the tip structure 1020 or at a location difficult to reach with a shockwave element angled at outward angle.

FIG. 10D illustrates a schematic, partial cross-sectional view of an alternative tip structure 1020a similar in some respects to the tip structure 1020 of FIGS. 10B-10C. Unlike the tip structure 1020 of FIGS. 10B-10C, the tapered portion 1041 of the tip structure 1020a is oriented at an outward and backward facing angle to propagate a shockwave from the shockwave element 530 in a generally backwards trajectory relative to the distal end 1021. This allows the shockwave element 530 to interact with the stone 1091 (FIG. 10A) when the stone 1091 is located behind the alternative tip structure 1020a. The tapered portion 1041 shown in FIG. 10D extends radially inward from the outer periphery 1027 of the end surface 1025 and towards the longitudinal axis 1005, while the tapered portion 1041 shown in FIGS. 10B-10C extend radially outward from the outer periphery 1027 and away from the longitudinal axis 1005.

Each outer tapered surface 1041 of tip structure 1020a may be part of a separate protrusion extending from the tip structure 1020a, a recess (e.g., groove) formed in the outer surface 1040, or part of the same lip that protrudes from the normal outer diameter of the tip structure 1020a. In some embodiments, each outer tapered surface 1041 may be part of separate recesses formed in the outer surface 1040. In some embodiments, the tapered portion 1041 may be perpendicular to the longitudinal axis 1005 such that the shockwave is propagated directly backwards (proximal) in a trajectory generally parallel with the longitudinal axis 1005. While not shown, the tip structure 1020a may also include the second array of shockwave elements 1132 arranged on the inner surface 1022.

The tapered outer surfaces 1041 of the tip structures 1020, 1020a may be disposed at different angles. For example, one or more the tapered outer surfaces 1041 may be oriented to direct the shockwaves in a generally forward trajectory as shown in FIG. 10B (e.g., angled to face away from a proximal end 1029 of the tip structure 1020), or in and a generally backwards trajectory as shown in FIG. 10D (e.g., angled to face toward the proximal end of the tip structure 1020a). In some embodiments, each tapered outer surface 1041 is disposed at a different angle relative to the distal end 1021. In some embodiments, such as shown in FIG. 10B-10C, the tip structure 1020 may have a plurality of tapered outer surfaces 1041 that are oriented at the same or substantially the same angle.

In embodiments where each tapered outer surface 1041 is disposed at a different angle relative to the distal end 1021, the operator may rotate the catheter to orient a desired shockwave element 530 on a specific tapered outer surface 1041 toward the kidney stone 1091. Additionally, the different angles of the tapered outer surface 1041 allow the operator to evaluate which shockwave element 530 associated therewith will achieve the desired result, such as a desired fragmentation or dislodgement.

Referring again to FIGS. 10B-10C, each shockwave element 530 in the first array of shockwave elements 1031 may be fired in unison or independently. Firing each shockwave element 530 independently is advantageous since some of the shockwave elements 530 may not be oriented towards the kidney stone 1091 and may instead be oriented at an anatomical feature of the kidney 1001. Thus, independent activation of the shockwave elements 530 in the first array 1031 facilitates selectively propagating shockwaves that are directed at the kidney stone 1091. In some embodiments, two or more of the shockwave elements 530 in the first array 1031 may be grouped together, and each individual group may be fired independently with each member of the group being fired in unison. In other embodiments, the shockwave elements 530 in the first array 1031 may be activated in series. In other embodiments, the shockwave elements 530 may be fired in unison. In some embodiments, the user may select the firing sequence for the shockwave elements 530.

The second array of shockwave elements 1032 is shown as including four shockwave elements 530 arranged with equidistant angular spacing along the first inner surface portion 1023. The second array of shockwave elements 1032, however, may comprise any number of shockwave elements 530 with equal or unequal angular spacing from each other. Additionally, one or more shockwave elements 530 may be arranged on the second inner surface portion 1024 in addition to one or more shockwave elements 530 being located at the first inner surface portion 1023. In some embodiments, one or more shockwave elements 530 may also be located on the planar surfaces 1028, such as each planar surface 1028 having a shockwave element 530.

The kidney stone 1091 (FIG. 10A) may be at least partially received within the portion of the lumen 1008 defined by the inner surface 1022 to position the stone 1091 adjacent the second array of shockwave elements 1032. The first inner surface portion 1023 is tapered to allow at least a portion of the object to be received within the tip structure 1020 while the second inner surface portion 1024 may have an inner diameter selected to inhibit large fragments from passing through the remainder of the catheter 1002 to avoid developing obstructions in the lumen 1008. Fluid may be aspirated through the lumen 1008 while the object is at least partially disposed within the tip structure 1020, such as when the shockwave elements 530 of the second array of shockwave elements 1032 are generating shockwaves. In some embodiments, the first array 1031 is not activated to produce shockwaves while the second array 1032 is being used to fragment the stone 1091. In other embodiments, one or more shockwave elements 530 in the first array 1031 may be activated to assist fragmentation of the kidney stone 191 while the first array 1031 is active.

In some embodiments, the first and second arrays of shockwave elements 1031, 1032 are part of a first set of shockwave elements. The catheter 1002 may include one or more second sets of shockwave elements, such as one or more shockwave elements 530 arranged on the second inner surface portion 1024 or arranged in the shaft of the catheter 1002 to inhibit the formation of clogs in the lumen 1008 downstream of the tip structure 1020.

In some embodiments, a kidney stone may be treatable using an articulatable scope that includes one or more shockwave elements to break up the stone. The scope with shockwave elements may be sufficient to fragment and remove the object without the aid of a catheter. However, the scope with shockwave elements may be used in combination with a catheter, such as being used in combination with a catheter including one or more shockwave elements.

FIG. 11 illustrates an example scope 1100 used in medical procedures in accordance with aspects of this disclosure. The scope 1100 can be a robotically or manually controlled instrument that can include an instrument base or handle 1110, an elongate shaft 1120, and an articulation section 1130. The elongate shaft 1120 can extend from the instrument base or handle 1110. The articulation section 1130 can include a proximal section 1140 and a distal section 1150. The distal end or tip of the distal section 1150 can include a tip portion 1172 (e.g., tip section) that includes one or more shockwave elements 530 configured to generate and propagate shockwaves at an object, such as a kidney stone, when the tip portion 1172 is disposed at a working position within a target area of the patient.

The scope 1100 also includes one or more articulation elements (e.g., control cables, pull wires, pull wire segments) that can be manipulated to actuate or deflect the articulation section into a desired pose. The articulation elements may run along the outer surface of the shaft 1120 and the articulation section 1130. Alternatively, the articulation elements can run along a working channel 1170 of the shaft 1120, or through a wall of the shaft 1120. The articulation elements can include one, two, three, four, five, six or more pull wires or segments.

Manipulation of the articulation elements can be controlled via one or more instrument drivers positioned within or connected to the instrument base or handle 1110. The instrument base 1110 can generally include an attachment interface having one or more mechanical inputs, e.g., receptacles, pulleys, or spools, which are designed to be reciprocally mated with one or more torque couplers on an attachment surface of an instrument driver. The instrument base 1110 can include a plurality of drive inputs. The articulation elements can be coupled to the drive inputs and extend along the shaft 1120. The drive inputs are configured to control or apply tension to the articulation elements in response to drive outputs from a tool driver of a medical robotic system. Furthermore, a robotic arm of a medical robotic system can be configured to maneuver and control the scope 1100. The robotic arm can be configured to apply tension to the articulation elements to bend the articulation section 1130.

In the embodiment illustrated in FIG. 11, the scope 1100 includes articulation elements 1160, 1162, 1164, 1166. The articulation elements 1160-1166 extend along the elongate shaft 1120 and terminate at the tip portion 1172. In some embodiments, the scope 1100 comprises a series of pulleys to which the articulation elements 1160-1166 can be operatively coupled that enable the shaft 1120 to translate relative to the handle 1110. The articulation elements 1160-1166 may be used to manipulate the scope 1100 into a desired pose. For example, the articulation elements 1160-1166 may be coordinated to place the tip portion 1172 in a working position in a target area of the patient, such as a specific calyx of a kidney. Additionally, the elongate shaft 1120 can rotate or roll in a direction about the longitudinal axis 1101. For example, the handle 1110 may be configured to rotate the elongate shaft 1120 about the longitudinal axis 1101 to orientate the tip portion 1172 in the desired working position.

In some embodiments, rather than being located on the end surface, as shown in FIG. 11, the shockwave elements 530 may be located on an inner surface of the working channel 1170 or the outer surface of the tip portion 1172. Any number of shockwave elements 530 can be included at the tip portion 1172, such as one, two, three, four, five, six, seven, or eight shockwave elements 530. The shockwave elements 530 may be arranged with equidistant or non-equidistant spacing, and may be formed on the same or different surfaces of the tip portion 1172. The shockwave elements 530 may be disposed at the same or different angles. For example, the shockwave elements 530 may be oriented such that the shockwaves propagate generally forward (e.g., directly forward, converging toward or diverging away from the longitudinal axis 1101), or generally backward (e.g., diverging away from the longitudinal axis. In some embodiments, the tip portion 1172 may include first and second arrays of shockwave elements, such as a first array arranged to generate and propagate the shockwaves in a generally forward trajectory towards the longitudinal axis 1101 while the second array may be arranged to generate and propagate the shockwaves at an angle away from the longitudinal axis 1101. In some embodiments, the scope 1100 may include one of the tip structures 520, 720, 820, 920, 1020, or 1020A described herein.

In some embodiments, one or more shockwave elements may be located along the working channel 1170 in a similar manner to the second set of one or more shockwave elements 730b of FIG. 7C. For example, the scope 1100 may include shockwave elements located along or proximal to the articulation section 1130 to help mitigate clogging of the working channel 1170 while fragments of the object are being aspirated through the working channel 1170.

In some embodiments, the scope 1100 can include an image capture device or camera 1184 and one or more light sources 1186 at the tip portion 1172 in addition to the shockwave elements 530. The light sources 1186 may comprise one or more light-emitting diodes or optical fibers configured to convey light from a remote light source (e.g., in a tower). The light sources 1186 can be configured to illuminate a target anatomy to facilitate visualization via images obtained with the camera 1184. For example, the camera 1184 and light sources 1186 can be used to help maneuver the tip portion 1172 adjacent to the object. The camera 1184 can also be used to observe the fragmentation process.

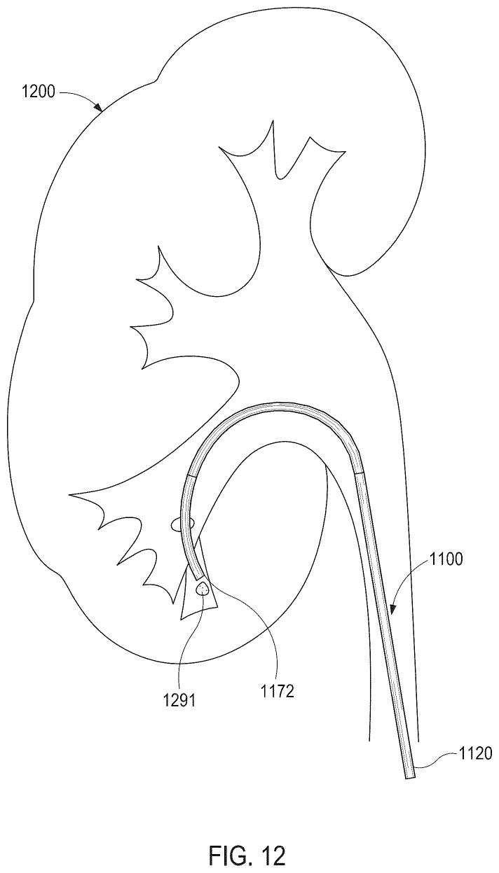

FIG. 12 illustrates the scope 1100 being used as a ureteroscope navigated through the anatomical pathway of a kidney 1200. Although the kidney 1200 is widely variable in shape, size, and configuration, it is roughly generalizable as a planar structure with an upper, middle, and lower pole. From each of these poles stem a series of calyces that point anteriorly or posteriorly. The scope 1100 can be a ureteroscope configured for insertion into a urinary tract of a patient. The scope 1100 can be rotated to roll the elongate shaft 1120 and thereby point or align the articulation section 1130 in the correct direction (orientation) to place the tip portion 1172 in a working position relative to a kidney stone 1201. The camera 1184 (FIG. 11) and light sources 1186 (FIG. 11) may be used to help visibly maneuver the tip portion 1172 into the working position. The shockwave elements 530 (FIG. 11) may be used to generate and propagate shockwaves to fragment the stone 1291.

Fragments of the stone 1291 may then be removed from the target area by aspirating fluid through the working channel 1170 (FIG. 11). In some embodiments, fragments may be removed using a basket tool configured to capture the fragments. The basket tool may form part of an end effector coupled to the tip portion 1172 or part of another tool inserted through the working channel 1170. In some embodiments, the fragments may be removed by aspiration through a catheter inserted into the patient through a percutaneous access device. In some embodiments, the catheter may not include shockwave elements or be otherwise configured to fragment the kidney stone 1291.

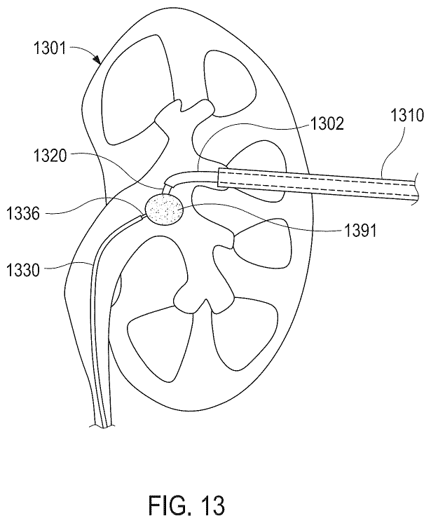

FIG. 13 illustrates treating a kidney stone 1391 within a kidney 1301 using a catheter 1302 inserted into the kidney 1301 through a percutaneous access-device 1310, and a ureteroscope 1330 inserted through the urethra, bladder, and ureter. The ureteroscope 1330 may be used to position the kidney stone 1391 in a working position for the catheter 1302. The catheter 1302 may also hold the kidney stone 1391 in position during the fragmentation process due to aspiration of fluid through the working channel of the catheter 1302. In some embodiments, the kidney stone 1391 may be fragmented by the catheter 1302, the ureteroscope 1330, or both the catheter 1302 and the ureteroscope 1330.

In some embodiments, both the catheter 1302 and ureteroscope 1330 may have one or more shockwave elements operable to fragment the kidney stone 1391. The catheter 1302 may have a tip structure 1320 that includes one or more shockwave elements 530 (FIGS. 5A-5C), such as having one or more arrays of shockwave elements 530. The catheter 1302 may comprise any of the catheters 140, 502, 602, and 1002 discussed herein. Additionally, the tip structure 1320 comprise any of the tip structures 520, 720, 820, 920, 1020, and 1020a described herein. In some embodiments, the ureteroscope 1330 may have a distal tip portion 1336 that includes one or more shockwave elements. In such embodiments, the ureteroscope 1330 may comprise the scope 1100 of FIG. 11.

In some embodiments, only one of the catheter 1302 and ureteroscope 1330 includes shockwave elements. As one example, the catheter 1302 may omit shockwave elements 530 while the ureteroscope 1330 includes shockwave elements configured to fragment the stone 1391. The catheter 1302 and/or the ureteroscope 1330 may be used to aspirate the fragments created by the shockwave elements of the ureteroscope. The catheter 1302 may be used to hold the kidney stone 1391 in position while the ureteroscope 1330 is used to fragment the stone 1391. As another example, the catheter 1302 may have one or more shockwave elements while the ureteroscope 1330 omits the shockwave elements. The ureteroscope 1330 may be used to position the kidney stone 1391 during fragmentation by the catheter 1302 and to remove one or more fragments, such as removing the fragments by aspiration.

As another example, the catheter 1302 may have one or more shockwave elements while the ureteroscope 1330 omits the shockwave elements but includes another element capable of fragmenting a stone, such as a ureteroscope with a laser filament or a working channel that allows another tool (e.g., a laser, a cutting instrument, lithotripter, etc.) to be inserted through the ureteroscope 1330 to fragment the kidney stone 1391. The catheter 1302 can be used to hold and aspirate the stone fragments during fragmentation. In some embodiments, the ureteroscope 1330 may omit any kind of fragmentation element and may be used to view and/or maneuver the stone 1391 for the catheter 1302.

In yet other embodiments, the ureteroscope 1330 may omit any type of device or mechanism operable to fragment the stone 1391, and the ureteroscope 1330 may instead be used to hold the stone 1391 or otherwise maintain the stone 1391 in a particular or known location. In such embodiments, the ureteroscope 1330 may be articulated and otherwise manipulated to pin down or isolate the stone 1391 at a known location. Once the stone 1391 is pinned down, the catheter 1302 with the shockwave elements may be advanced to the stone 1391 and operated to fragment the stone 1391. Alternatively, in such embodiments, the ureteroscope 1330 may provide a working channel for the deployment of a basket operable to capture and hold the stone 1391. Once the stone 1391 is captured, the catheter 1302 with the shockwave elements may be advanced to the stone 1391 and operated to fragment the stone 1391.

In some embodiments, an object may be fragmented by inserting a probe equipped with one or more shockwave elements through another medical device, such as the working channel of a scope or catheter. Utilizing a probe reduces the cost of the catheter and/or scope since neither have shockwave elements. Additionally, the probe can be used with conventional scopes and/or catheters since these devices provide a pathway for the probe to reach the object. However, the probe can be used in conjunction with a catheter and/or scope that include the shockwave elements, such as when fragmenting a large stone.

FIG. 14A illustrates treating a kidney stone 1491 within a kidney 1401 using a catheter 1402 inserted through a percutaneous access-device 1410 and a ureteroscope 1430 inserted into the kidney 1041. The ureteroscope 1430 may reach the kidney 1401 through the urethra, bladder, and ureter. The catheter 1402, however, does not include shockwave elements at a distal tip thereof. Instead, a probe 1420 is inserted through a working channel (e.g., lumen) of the catheter 1402 and includes one or more shockwave elements 1424.

FIG. 14B illustrates a schematic side view of the probe 1420. The probe 1420 includes an elongate shaft 1422 and one or more shockwave elements 1424 are provided at a distal end 1423 of the shaft 1422. The shaft 1422 may be formed of a flexible material that allows the probe 1420 to be inserted through the working channel of the catheter 1402 once the catheter 1402 has been manipulated into a desired pose to direct the distal end 1423 toward the kidney stone 1491.

In some embodiments, the shockwave elements 1424 are positioned and oriented to propagate shockwaves in a direction generally parallel to a longitudinal axis 1421 of the probe 1420 as, shown schematically by the arrow 1425. The shockwave elements 1424 may be similar to the shockwave element 530 (FIGS. 5A-5C). For example, each shockwave element 1424 may be an electrode pair used to create a plasma arc that forms the bubble, a laser element used to heat up the fluid to create a bubble, or a piezoelectric element that changes shape in response to an electric current to propagate shockwaves toward the object.

The probe 1420 may be used to fragment the stone 1491 while fragments thereof are removed by the ureteroscope 1430, such as being aspirated through a working channel of the ureteroscope 1430. In some embodiments, a distal tip portion 1336 of the ureteroscope 1430 may include one or more shockwave elements (e.g., shockwave elements 530 of FIG. 11) or other elements (e.g., laser filament) capable of fragmenting the kidney stone 1491 to facilitate fragmentation of the kidney stone 1491 with the probe 1420 disposed in the catheter 1402 and the ureteroscope 1430. For example, the kidney stone 1491 may be simultaneously fragmented with the probe 1420 and the ureteroscope 1430.

In some embodiments, the probe 1420 may instead be inserted through a working channel of the ureteroscope 1430 rather than the catheter 1402, such as inserting the probe 1420 once the ureteroscope 1430 has been manipulated into a desired pose. In such embodiments, the probe 1420 may be used to fragment the stone 1491 while the fragments are aspirated through the catheter 1402. In some embodiments, the catheter 1402 may include one or more shockwave elements or other elements capable of fragmenting the kidney stone 1491 so that the catheter 1402 and the probe 1420 disposed in the ureteroscope 1430 can both fragment the kidney stone 1491 simultaneously.

In some embodiments, the probe 1420 may be inserted into a catheter or scope that includes one or more shockwave elements. This allows the probe 1420 to assist in the fragmentation with the shockwave elements of the catheter or scope. Additionally, the probe 1420 may be inserted through the catheter or scope that includes the shockwave elements in the event that the shockwave elements of the catheter or scope fail or malfunction.

In one embodiment, a method of fragmenting an object at a target site within a patient comprises positioning a medical device in a working position adjacent to the object within the patient; inserting a probe through a lumen of the medical device, the probe including one or more shockwave elements provided at a distal tip of the probe; and fragmenting the object with the one or more shockwave elements. The medical device comprises either a catheter disposed in a percutaneous access device or a scope.

In one or more embodiments, a scope comprises an elongate shaft including a lumen and one or more articulation elements configured to change the pose of the elongate shaft; and one or more shockwave elements disposed at a distal tip portion of the elongate shaft and configured to generate (propagate) shockwaves to fragment an object located at a target site within a patient.

### Distal Tip Structures with Shockwave Elements

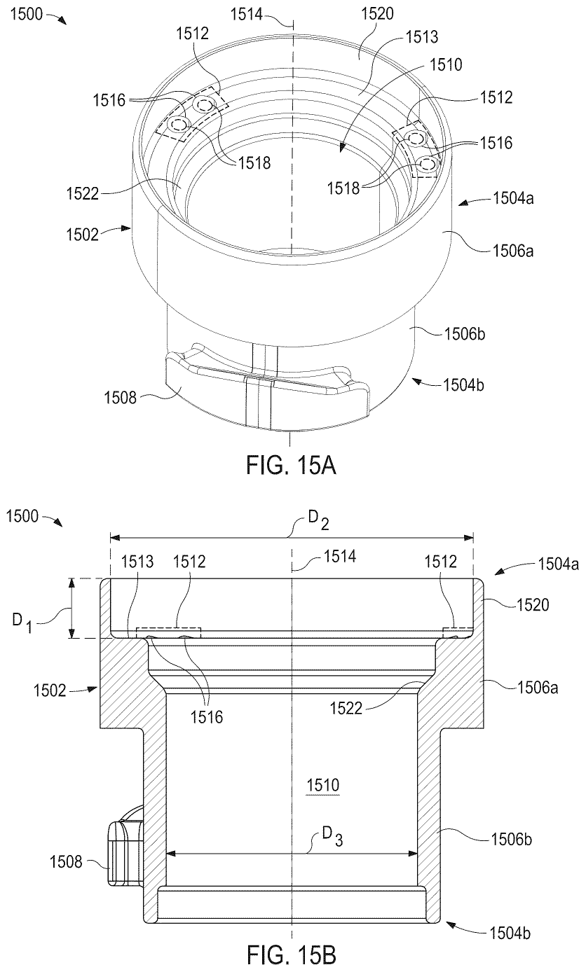

FIGS. 15A and 15B are isometric and cross-sectional side views, respectively, of an example tip structure 1500, according to one or more embodiments of the present disclosure. The tip structure 1500 may be similar in some respects to any of the tip structures 520, 720, 820, 920, 1020, 1020a described herein. For instance, the tip structure 1500 may be configured to be operatively coupled to the distal end of a shaft of any of the catheters or percutaneous access devices (collectively referred to herein as “catheters”) disclosed herein. In other embodiments, however, the tip structure 1500 may form an integral part or extension of the distal end of the shaft, without departing from the scope of the disclosure. In such embodiments, the tip structure 1500 may not constitute a separate component part, but may instead form an extension of the corresponding shaft.

As illustrated, the tip structure 1500 provides a generally cylindrical body 1502 having a first or “distal” end 1504a and an opposing second or “proximal” end 1504b. While the body 1502 is shown as having a substantially cylindrical form, the tip structure 1500 may assume other forms, such as a polygonal shape (e.g., triangular rectangular, square, hexagonal, octagonal, etc.). In embodiments where the tip structure 1500 is a separate component part, the proximal end 1504b may be sized and otherwise configured to mate with the distal end of a catheter or percutaneous access device. More specifically, the body 1502 may provide a first or “distal” portion 1506a and a second or “proximal” portion 1506b extending from the distal portion 1506a. The diameter of the proximal portion 1506b may be smaller than the diameter of the distal portion 1506a, thereby enabling the body 1502 to be operatively coupled and “mated” to the distal end of the catheter or percutaneous access device at the proximal end 1504b. The tip structure 1500 may be attached to the shaft, for example, using at least one of an adhesive, a fastener, an interlocking mechanism (e.g., tabs, grooves, etc.), or any combination thereof.

In some embodiments, as illustrated, one or more cable mounts 1508 may be provided on the body 1502 at or near the proximal end 1504b. The cable mounts 1508 may be sized and otherwise configured to receive corresponding articulation elements (e.g., articulation elements 714) such that antagonistic operation (e.g., pulling) of the articulation elements will cause the tip structure 1500 and the distal end of the catheter or percutaneous access device to articulate.

The body 1502 defines an interior 1510 that extends between the distal and proximal ends 1504a, b and is configured to communicate with the lumen (e.g., lumen 508) of the catheter or percutaneous access device to which the tip structure 1500 is operatively coupled.

One or more shockwave elements 1512 (shown as dashed boxes) are mounted to the body 1502 at the distal end 1504a and generally at the opening to the interior 1510. In the illustrated embodiment, the distal end 1504a defines a generally planar and circular distal surface 1513, and the shockwave elements 1512 are mounted to the body 1502 at the distal surface 1513. In some embodiments, as illustrated, the distal surface 1513 may extend substantially perpendicular to a central axis 1514 of the tip structure 1500. In other embodiments, however, the distal surface 1513 may be angled and offset from perpendicular to the central axis 1514, without departing from the scope of the disclosure.

The shockwave elements 1512 may be the same as or similar to the shockwave elements 530 described herein, and therefore may operate in substantially the same way to generate shockwaves to promote fragmentation of objects (e.g., kidney stones). In the illustrated embodiment with the distal surface 1513 extending substantially perpendicular to the central axis 1514, the shockwave elements 1512 are oriented to propagate shockwaves in a direction substantially parallel to the central axis 1514. In other embodiments, angling the distal surface 1514 relative to the central axis 1514 may result in the orientation of the shockwave elements 1512 being varied, thereby resulting in the propagation of shockwaves in directions offset from parallel to the central axis 1514.

In the illustrated embodiment, a plurality of internal lumens 1516 are defined in the sidewall of the body 1502 and terminate at the distal end 1504a. As best seen in FIG. 15A, the internal lumens 1516 may be configured to convey corresponding connections or “lead wires” 1518 (shown in dashed lines) to the shockwave elements 1512 and thereby provide electrical communication (power) therewith for operating the shockwave elements 1512. Two internal lumens 1516 allow for functionality when the shockwave element 1512 incorporates positive and negative leads; e.g., an electrode pair makes up a single shockwave element 1512 or “emitter”. Running the lead wires 1518 in separate internal lumens 1516 helps avoid shorts between the lead wires 1518. In some embodiments, a third internal lumen 1516 may be included and shared with an angularly adjacent shockwave elements 1512. In such embodiments, each shockwave element 1512 will include a shared electrode. In yet other embodiments, a single internal lumen 1516 may be used when the shockwave element 1512 is a laser element, and the connection 1518 may comprise a fiber optic filament that guides a laser beam that exits the shockwave element 1512, which may be the end of the fiberoptic filament.

The illustrated embodiment includes three shockwave elements 1512 (one occluded) attached to the tip structure 1500. In other embodiments, however, more or less than three shockwave elements 1512 may be included, without departing from the scope of the disclosure. Moreover, as illustrated, the shockwave elements 1512 are angularly spaced from each other about the circumference of the body 1502 at the distal end 1504a. The shockwave elements 1512 may be equidistantly or non-equidistantly spaced from each other.

According to embodiments of the present disclosure, the tip structure 1500 may further provide a standoff feature 1520 extending distally from the distal end 1504a of the body 1502, such as from the distal surface 1513. In some embodiments, as illustrated, the standoff feature 1520 forms an integral part or extension of the body 1502. In other embodiments, however, the standoff feature 1520 may be coupled or otherwise attached to the body 1502 at the distal end 1504a. In such embodiments, the standoff feature 1520 may be made of a material different from the body 1502, but could alternatively be made of the same material.

In the illustrated embodiment, the standoff feature 1520 comprises an annular wall extending distally from the distal end 1504a of the body 1502. In some embodiments, as illustrated, the standoff feature 1520 extends substantially parallel to the central axis 1514. In other embodiments, however, the standoff feature 1520 may extend from the distal end 1504a at an angle offset from parallel to the central axis 1514, without departing from the scope of the disclosure. Moreover, while shown as a generally circular-shaped feature, the standoff feature 1502 may alternatively exhibit other shapes, such as polygonal (e.g., rectangular, square, etc.) or custom-shaped.

As best seen in FIG. 15B, the standoff feature 1520 extends a distance D₁ past (beyond) the distal end 1504a and otherwise past where the shockwave elements 1512 are mounted. The distance D₁ may be at least 0.1 mm, but could range between about 0.1 mm and about 5 mm, without departing from the scope of the disclosure. Moreover, the standoff feature 1520 exhibits an inner diameter D₂ that is larger than the inner diameter D₃ of the interior 1510. Accordingly, the distal surface 1513 and the shockwave elements 1512 are accommodated within the inner diameter D₂.

In some embodiments, as illustrated, a radial shoulder 1522 may be provided or otherwise defined within the interior 1510. The radial shoulder 1522 generally provides a transition between the distal surface 1513 and the interior 1510, and provides a reduced diameter section as compared to the diameter of the interior 1510 at the distal surface 1513. The reduced diameter may be advantageous in helping to inhibit objects or fragments larger than a predetermined size from entering the interior 1510. Accordingly, objects larger than the diameter of the radial shoulder 1522 will be prevented from entering and clogging the lumen (e.g. the lumen 508) of the catheter (or percutaneous access device) to which the tip structure 1500 is attached (or forms part of). In the illustrated embodiment, the radial shoulder 1522 is beveled and otherwise angled relative to the central axis 1514. This may prove advantageous in easing fragmented objects and particulate matter into the interior 1510. In other embodiments, however, the radial shoulder 1522 may extend orthogonally to the central axis 1514, without departing from the scope of the disclosure.

The standoff feature 1520 helps provide a gap or distance between the shockwave elements 1512 and an adjacent object (e.g., a kidney stone) to be fragmented by the shockwave elements 1512. More specifically, in applications where the object is larger than the inner diameter D₂ of the standoff feature, instead of advancing the tip structure 1500 until the shockwave elements 1512 come into direct contact with the object, the standoff feature 1520 directly contacts the object and thereby generates a separation gap between the shockwave elements 1512 and the adjacent object. The separation gap helps prevent the shockwave elements 1512 from directly contacting the adjacent object, which may help in the creation of more forceful cavitation bubbles used to fragment the object. More specifically, the created separation gap may ensure that the shockwave elements 1512 maintain optimal energy, focus, and pressure to fracture the target efficiently and safely. If the shockwave elements 1512 are too close to the target, the formation and explosion (cavitation) of bubbles may be less efficient. In contrast, if the emitter is too far, the energy may be too weak to fracture the target effectively. Moreover, the standoff feature 1520 may also prove advantageous in protecting surrounding anatomy from stray laser radiation or jettisoned fragments. The standoff feature 1520 could also function as structural support.

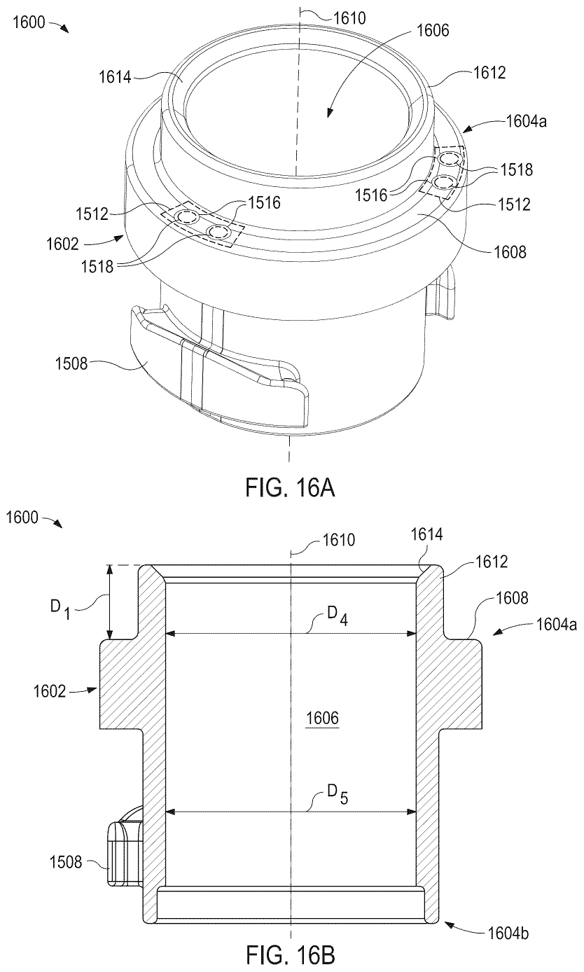

FIGS. 16A and 16B are isometric and cross-sectional side views, respectively, of another example tip structure 1600, according to one or more additional embodiments of the present disclosure. The tip structure 1600 may be similar in some respects to the tip structure 1500 of FIGS. 15A-15B, and therefore may be best understood with reference thereto, where like numerals will correspond to similar components not described again in detail. Similar to the tip structure 1500, for example, the tip structure 1600 may be configured to be operatively coupled to the distal end of a shaft of any of the catheters or percutaneous access devices disclosed herein. In other embodiments, however, the tip structure 1600 may form an integral part of the distal end of the shaft, without departing from the scope of the disclosure.

As illustrated, the tip structure 1600 provides a generally cylindrical body 1602 having opposing distal and proximal ends 1604a, b. The proximal end 1604b may be sized and otherwise configured to mate with the distal end of a catheter or percutaneous access device, and the cable mounts 1508 may be provided on the body 1602 at or near the proximal end 1604b. The body 1604 defines an interior 1606 that extends between the distal and proximal ends 1604a, b and is configured to communicate with the lumen (e.g., lumen 508) of the catheter or percutaneous access device to which the tip structure 1600 is operatively coupled.

One or more shockwave elements 1512 (shown as dashed boxes) are mounted to the body 1602 at the distal end 1604a and, more particularly, mounted to a generally planar and circular distal surface 1608 provided at the distal end 1604a. The distal surface 1608 may extend substantially perpendicular to a central axis 1610 of the tip structure 1600, but could alternatively be angled and offset from perpendicular to the central axis 1610, without departing from the scope of the disclosure. As best seen in FIG. 16A, the plurality of internal lumens 1516 are defined in a sidewall of the body 1602 and terminate at the distal end 1604a. Corresponding lead wires 1518 (shown in dashed lines) extend within the internal lumens 1516 and terminate at the shockwave elements 1512 to provide electrical communication therewith for operating the shockwave elements 1512.

Three shockwave elements 1512 (one occluded) are included in the tip structure 1600, but more or less than three shockwave elements 1512 may be included, without departing from the scope of the disclosure. Moreover, as illustrated, the shockwave elements 1512 are angularly and equidistantly spaced from each other about the circumference of the body 1602 at the distal end 1604a, but could alternatively be non-equidistantly spaced.

Similar to the tip structure 1500 of FIGS. 15A-15B, the tip structure 1600 may further provide a standoff feature 1612 extending distally from the distal end 1604a of the body 1602, such as from the distal surface 1608. In some embodiments, as illustrated, the standoff feature 1612 forms an integral part or extension of the body 1602. In other embodiments, however, the standoff feature 1612 may be coupled or otherwise attached to the body 1602 at the distal end 1604a. In such embodiments, the standoff feature 1612 may be made of the same or different material from the body 1602.

In the illustrated embodiment, the standoff feature 1612 comprises an annular wall extending distally from the distal end 1604a of the body 1602. In some embodiments, as illustrated, the standoff feature 1612 may extend substantially parallel to the central axis 1610. In other embodiments, however, the standoff feature 1612 may extend from the distal end 1604a at an angle offset from parallel to the central axis 1610, without departing from the scope of the disclosure.

The standoff feature 1612 is provided such that the distal surface 1608 and the shockwave elements 1512 are arranged radially outside the annular wall of the standoff feature 1612. As best seen in FIG. 16B, the standoff feature 1612 extends the distance D₁ past (beyond) the distal surface 1608. Moreover, the standoff feature 1612 exhibits an inner diameter D₄ that is the same as the inner diameter D₅ of the interior 1606. Accordingly, the standoff feature 1612 effectively extends the axial length of the interior 1606 at the same diameter D₄, D₅.

In some embodiments, as illustrated, the standoff feature 1612 may define otherwise provide a beveled distal surface 1614 that leads into the interior 1610. The beveled distal surface 1614 may be advantageous easing fragmented objects and particulate matter into the interior 1606. In other embodiments, however, the beveled distal surface 1614 may extend orthogonal to the central axis 1610, without departing from the scope of the disclosure. In yet other embodiments, the beveled distal surface 1614 may be beveled toward the exterior of the tip structure 1600.

The purpose and advantages of the standoff feature 1612 may be the same as the purpose and advantages of the standoff feature 1520 of FIGS. 15A-15B, and therefore will not be discussed again in detail.

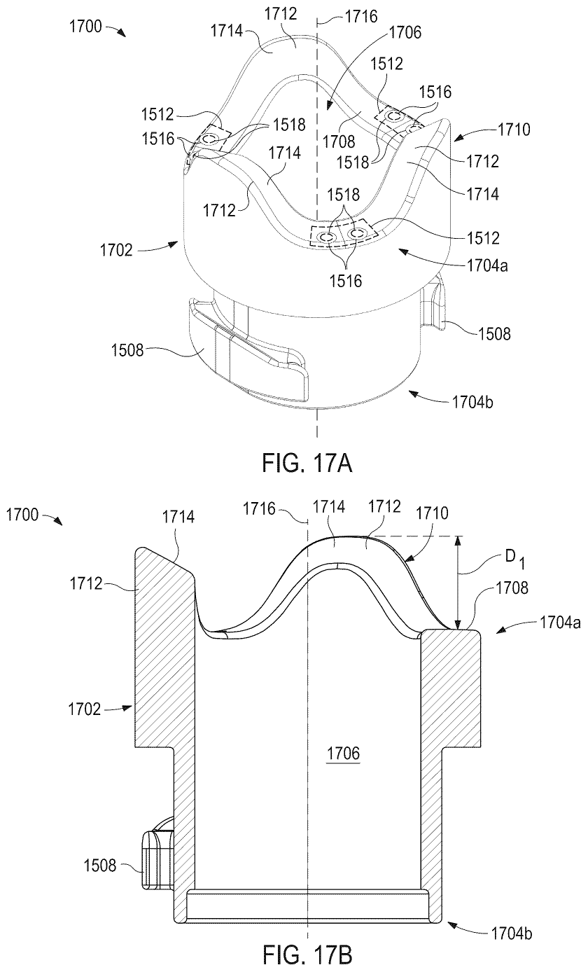

FIGS. 17A and 17B are isometric and cross-sectional side views, respectively, of another example tip structure 1700, according to one or more additional embodiments of the present disclosure. The tip structure 1700 may be similar in some respects to the tip structures 1500 and 1600 of FIGS. 15A-15B and 16A-16B, respectively, and therefore may be best understood with reference thereto, where like numerals will correspond to similar components not described again in detail. Similar to the tip structures 1500, 1600, for example, the tip structure 1700 may be configured to be operatively coupled to the distal end of a shaft of any of the catheters or percutaneous access devices disclosed herein. In other embodiments, however, the tip structure 1700 may form an integral part of the distal end of the shaft, without departing from the scope of the disclosure.

As illustrated, the tip structure 1700 provides a generally cylindrical body 1702 having opposing distal and proximal ends 1704a, b. The proximal end 1704b may be sized and otherwise configured to mate with the distal end of a catheter or percutaneous access device, and the cable mounts 1508 may be provided on the body 1702 at or near the proximal end 1704b. The body 1704 defines an interior 1706 that extends between the distal and proximal ends 1704a, b and is configured to communicate with the lumen (e.g., lumen 508) of the catheter or percutaneous access device to which the tip structure 1700 is operatively coupled.

One or more shockwave elements 1512 (shown as dashed boxes) are mounted to the body 1702 at the distal end 1704a and, more particularly, mounted to a distal surface 1708 provided at the distal end 1704a. As best seen in FIG. 17A, the plurality of internal lumens 1516 are defined in a sidewall of the body 1702 and terminate at the distal end 1704a. Corresponding lead wires 1518 (shown in dashed lines) extend within the internal lumens 1516 and terminate at the shockwave elements 1512 to provide electrical communication therewith for operating the shockwave elements 1512. While three shockwave elements 1512 are included in the tip structure 1700, more or less than three shockwave elements 1512 may be included, without departing from the scope of the disclosure.

Similar to the tip structures 1500 and 1600 of FIGS. 15A-15B and 16A-16B, the tip structure 1700 may further provide a standoff feature 1710 extending distally from the distal end 1704a of the body 1702, such as from the distal surface 1708. Unlike the tip structures 1500 and 1600, however, the standoff feature 1710 comprises an undulating or castellated feature. More specifically, the standoff feature 1710 comprises a plurality of lobes 1712 angularly spaced from each other about the circumference of the body 1702 at the distal surface 1708 and extending distally from the distal surface 1708. Each lobe 1712 extends the distance D₁ past (beyond) the distal surface 1708.

In the illustrated embodiment, the tip structure 1700 includes three lobes 1712, and a single shockwave element 1512 interposes angularly adjacent lobes 1712. Accordingly, the shockwave elements 1512 may be radially aligned with the lobes 1712, such as in the same radial track (plane). In other embodiments, however, the tip structure 1700 may include more or less than three lobes 1712, and more than one shockwave element 1512 may interpose angularly adjacent lobes 1712. In yet other embodiments, no shockwave element 1512 may be present between one or more of the angularly adjacent lobes 1712, without departing from the scope of the disclosure.

In some embodiments, as illustrated, the lobes 1712 may be curved and otherwise arcuate, and thereby provide a smooth and curved transition between the distal surface 1708 and the corresponding lobes 1712. In other embodiments, however, one or more of the lobes 1712 may exhibit polygonal features, such as including sharp corners. In such embodiments, the transition between the distal surface 1708 and the corresponding lobe 1712 may be abrupt, such as at a 90° angle or other sharp angles.

In some embodiments, as illustrated, each lobe 1712 may provide a beveled distal surface 1714 that leads into the interior 1706. The beveled distal surface 1714 may be advantageous easing fragmented objects and particulate matter into the interior 1706. In other embodiments, however, the beveled distal surface 1714 may extend orthogonal to a central axis 1716 of the body 1702, without departing from the scope of the disclosure. In yet other embodiments, the beveled distal surface 1714 may lead to the exterior of the body 1702.

In some embodiments, as illustrated, the lobes 1712 form integral parts or extensions of the body 1702. In other embodiments, however, the lobes 1712 may be coupled or otherwise attached to the body 1702 at the distal end 1704a. In such embodiments, the standoff feature 1710 may be made of a material that is the same or different from the body 1702. In some embodiments, as illustrated, the lobes 1712 may extend substantially parallel to the central axis 1716. In other embodiments, however, the lobes 1712 may extend from the distal end 1704a at an angle offset from parallel to the central axis 1716, without departing from the scope of the disclosure.

The purpose and advantages of the standoff feature 1710 may be the same as the purpose and advantages of the standoff features 1520 and 1612 of FIGS. 15A-15B and FIGS. 16A-16B, and therefore will not be discussed again in detail.

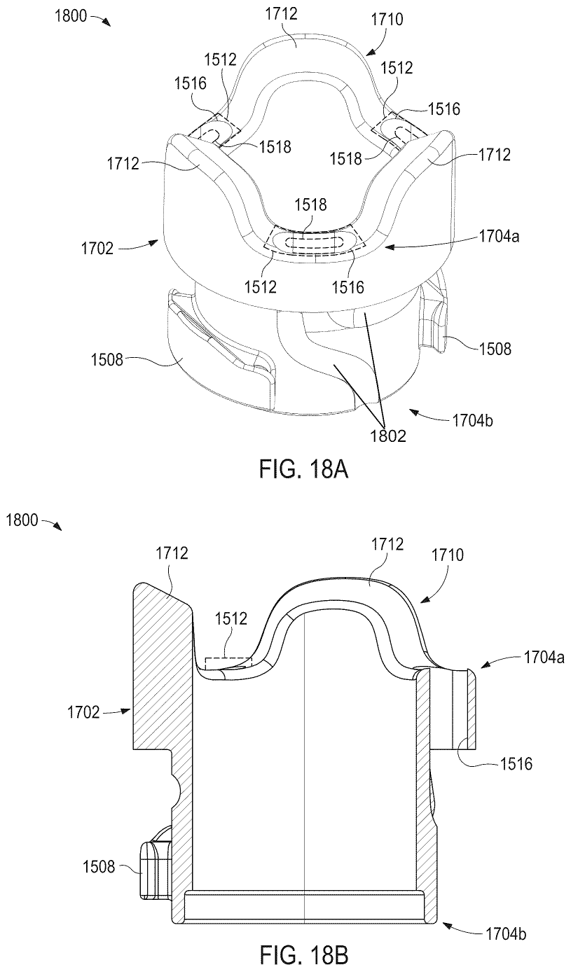

FIGS. 18A and 18B are isometric and cross-sectional side views, respectively, of another example tip structure 1800, according to one or more additional embodiments of the present disclosure. The tip structure 1800 may be similar in some respects to the tip structure 1700 of FIGS. 17A-17B, and therefore may be best understood with reference thereto, where like numerals will correspond to similar components not described again in detail. Similar to the tip structure 1700, for example, the tip structure 1800 includes the body 1702 with opposing distal and proximal ends 1704a, b, and the cable mounts 1508 are provided on the body 1702 at or near the proximal end 1704b. The body 1704 defines the interior 1706, and one or more shockwave elements 1512 (shown as dashed boxes) are mounted to the body 1702 at the distal end 1704a and, more particularly, to the distal surface 1708. Moreover, similar to the tip structure 1700, the tip structure 1800 includes the standoff feature 1710 with the plurality of lobes 1712 angularly spaced from each other about the circumference of the body 1702 and extending from the distal surface 1708.

Unlike the tip structure 1700 of FIGS. 17A-17B, however, a single internal lumen 1516 is defined in a sidewall of the body 1702 to correspond with each shockwave element 1512. Moreover, a single connection 1518 (shown in dashed lines) extends within each internal lumen 1516 and terminates at a corresponding one of the shockwave elements 1512 to provide electrical communication therewith for operating the shockwave elements 1512. The single internal lumens 1516 may help in manufacturing (assembly) to enable the tolerance and flexibility of the corresponding shockwave elements 1512 to be installed together easier than into individual holes. In such embodiments, installing the shockwave elements 1512 may include a potting step to keep them in position and separated. Moreover, as illustrated, the body 1702 may provide or define channels 1802 to convey the connections 1518 to the internal lumens 1516, and also to help provide alignment as well wiring the shockwave elements 1512 in series.

In one embodiment, a tip structure for a catheter includes a cylindrical body having opposing distal and proximal ends and defining an interior that extends between the distal and proximal ends, a distal surface provided at the distal end, one or more shockwave elements mounted to the body at the distal surface and operable to generate shockwaves to fragment an object, and a standoff feature extending distally from the distal surface to form a separation gap between the one or more shockwave elements and the object to be fragmented.

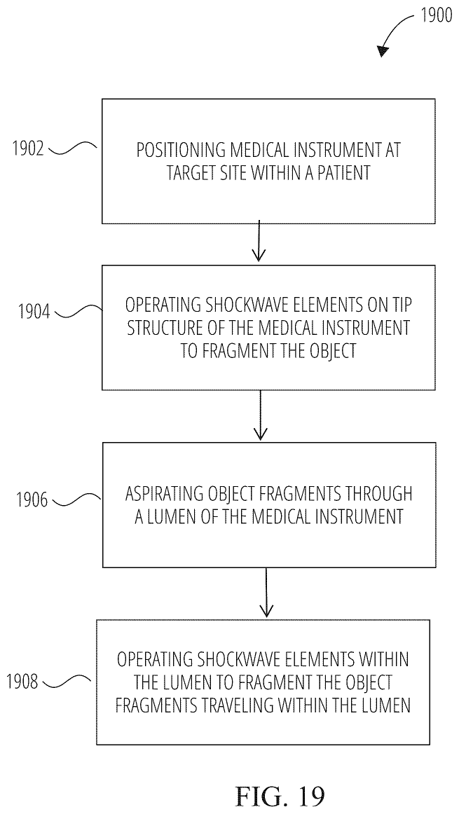

FIG. 19 is a flow chart of an example method 1900 of fragmenting an object located at a target site (e.g., kidney) within a patient using a medical instrument described herein, such as catheter 502 of FIG. 5A. The method 1900 may be conducted as a fully automated surgical procedure performed by the robotic medical system 100, a partial automated surgical procedure including both automated and manual operations, or a fully manual procedure.

At operation 1902, the medical instrument is positioned advanced into and within a target site within the patient to place a tip structure (e.g., tip structure 520; FIG. 5A) of the medical instrument adjacent the object. The tip structure includes one or more shockwave elements (e.g., shockwave elements 530, FIG. 5B) configured to propagate shockwaves to fragment the object. In some embodiments, the control system 150 (FIG. 1) may instruct the robotic system 110 to position the distal tip at the target site, such as by inserting the medical device through the percutaneous access device 510 (FIG. 5A). The control system 150 may operate the robotic system 110 in response to instructions input by the physician 160 (FIG. 1). The medical instrument may instead be positioned manually.

At operation 1904, and after the medical instrument reaches the desired position, the shockwave elements are operated (actuated) to propagate shockwaves toward the object. In some embodiments, the control system 150 causes the shockwave elements to propagate the shockwaves by selectively supplying current from a generator.

At operation 1906, fragments of the object are aspirated through a lumen within the medical instrument. The control system 150 may activate the aspiration system 170 (FIG. 1) to provide the aspiration, for example. In some embodiments, operation 1906 may occur prior to or simultaneously with operation 1904. For example, aspiration through the lumen may retain the object in position against the tip structure which helps direct the shockwaves directly at the object.

At operation 1908, object fragments traveling through the lumen are further fragmented using additional shockwave elements (e.g., shockwave elements 530, FIG. 7) arranged within the lumen to inhibit clogging. In some embodiments, these additional shockwave elements are operated by the control system 150 which may selectively supply current from the generator to cause the shockwave elements to propagate the shockwaves.

Embodiments disclosed herein include:
A. A catheter, including an elongate shaft having opposing proximal and distal ends; a tip structure coupled to the distal end of the elongate shaft and including an end surface at a distal end of the tip structure, an inner surface defining a lumen terminating at the distal end of the tip structure, and an outer surface opposite the inner surface; and one or more elements arranged on the inner surface and configured to propagate shockwaves to fragment an object.
B. A catheter including an elongate shaft having opposing proximal and distal ends and including an inner surface defining a lumen extending at least partially between the proximal and distal ends; and one or more elements arranged on the inner surface and configured to propagate shockwaves to fragment one or more objects present within the lumen.
C. A catheter including an elongate shaft having opposing proximal and distal ends; a tip structure arranged at the distal end and including an end surface at a distal end of the tip structure, an inner surface defining a lumen and terminating at the distal end of the tip structure, an inner surface defining a lumen and terminating at the distal end of the tip structure, wherein the inner surface includes a first inner surface portion extending from the distal end of the tip structure and exhibiting a variable inner diameter and a second inner surface portion extending from the first inner surface portion and having a substantially constant inner diameter; an outer surface opposite the inner surface; and one or more elements arranged on the second inner surface portion and configured to propagate shockwaves to fragment an object located within the tip structure.
D. A tip structure for a catheter including a cylindrical body having opposing distal and proximal ends and defining an interior that extends between the distal and proximal ends; a distal surface provided at the distal end; one or more elements mounted to the cylindrical body at the distal surface and configured to generate shockwaves to fragment an object; and a standoff feature extending distally from the distal surface to form a separation gap between the one or more elements and the object to be fragmented.

Element 1: wherein the one or more elements are selected from the group consisting of an electrode pair, a laser element, and a piezoelectric element. Element 2: wherein the one or more elements comprise one or more first elements, the catheter further comprising one or more second elements positioned on an inner surface of the elongate shaft and configured to propagate shockwaves to fragment one or more objects located within the elongate shaft. Element 3: wherein the inner surface of the tip structure includes: a first inner surface portion extending proximally from the distal end of the tip structure and tapering at an angle toward a longitudinal axis of the tip structure; and a second inner surface portion extending from the first inner surface portion, wherein the one or more elements are arranged on the first inner surface portion. Element 4: wherein the inner surface of the tip structure includes: a first inner surface portion extending proximally from the distal end of the tip structure; and a second inner surface portion extending from the first inner surface portion proximally to the proximal end, wherein the one or more elements are arranged on the first inner surface portion. Element 5: wherein the one or more elements comprise one or more first elements, the catheter further comprising one or more second elements arranged on the second inner surface portion to propagate shockwaves to fragment the object. Element 6: wherein the one or more elements comprise a first array of elements, the catheter further comprising a second array of elements arranged on the outer surface of the tip structure to propagate shockwaves. Element 7: wherein the outer surface comprises a plurality of outer tapered surfaces oriented to face away from the lumen, and wherein each shockwave element of the second array of elements is disposed on a corresponding one of the plurality of outer tapered surfaces. Element 8: wherein a number of outer tapered surfaces is equivalent to a number of elements of the second array of elements. Element 9: wherein each outer tapered surface is oriented such that shockwaves produced by the second array of elements are propagated in a forward trajectory away from the distal end of the tip structure. Element 10: wherein each outer tapered surface is oriented parallel to a longitudinal axis of the tip structure such that shockwaves produced by the second array of elements are propagated in a trajectory perpendicular to the longitudinal axis. Element 11 wherein each outer tapered surface is orientated at an outward and backward facing angle such that shockwaves produced by the second array of elements are propagated in a backward trajectory. Element 12: further comprising a standoff feature at the distal end extending distally from a portion of the inner surface where the one or more elements are arranged to form a separation gap between the one or more elements and the object to be fragmented. Element 13: wherein the tip structure further comprising a body, wherein the standoff feature extends from a distal end of the body, and wherein the one or more elements are arranged on a distal surface of the body. Element 14: wherein the one or more elements are selected from the group consisting of an electrode pair, a laser element, and a piezoelectric element. Element 15: wherein the one or more elements are arranged at a same axial position along the elongate shaft. Element 16: wherein the one or more elements comprise one or more first elements, and the inner surface comprises a first inner surface, the catheter further comprising: a tip structure arranged at the distal end and including an end surface at a distal end of the tip structure and a second inner surface defining a portion of the lumen extending through the tip structure and terminating at the distal end of the tip structure; and one or more second elements arranged on the second inner surface. Element 17: further comprising one or more third elements arranged on an outer surface of the tip structure. Element 18: wherein the one or more elements comprise one or more first elements, the catheter further comprising one or more second elements arranged on an inner surface of the elongate shaft and being configured to fragment one or more objects located within the elongate shaft. Element 19: wherein the one or more elements comprise one or more first elements, the catheter further comprising one or more second elements arranged on the first inner surface portion configured to propagate shockwaves. Element 20: further comprising one or more third elements arranged on the outer surface of the tip structure to propagate shockwaves away from the outer surface. Element 21: wherein the one or more elements are selected from the group consisting of an electrode pair, a laser element, and a piezoelectric element. Element 22: wherein the distal surface is planar and extends substantially perpendicular to a central axis of the body. Element 23: further comprising: a plurality of internal lumens defined in a sidewall of the body and terminating at the distal end; and one or more lead wires arranged within each internal lumen and operatively coupled to a corresponding one of the one or more elements to provide electrical power thereto. Element 24: the one or more elements comprise a plurality of elements that are angularly spaced from each equidistantly about a circumference of the distal surface. Element 25: wherein the standoff feature forms an integral part and extension of the body. Element 26: wherein the standoff feature comprises a separate component part from the body and is operatively coupled to the body at the distal end. Element 27: wherein the standoff feature comprises an annular wall extending distally from the distal surface. Element 28: wherein the standoff feature extends substantially parallel to a central axis of the body. Element 29: wherein the standoff feature extends a distance beyond the distal surface, and wherein the distance ranges between about 1 mm and about 5 mm. Element 30: wherein the standoff feature exhibits an inner diameter larger than an inner diameter of the interior, and wherein the distal surface and the one or more elements are positioned within the inner diameter of the standoff feature. Element 31: further comprising a radial shoulder defined within the interior and providing a reduced diameter section within the interior, wherein the diameter of the interior distal to the radial shoulder is greater than the diameter of the interior proximal to the radial shoulder. Element 32: wherein the radial shoulder is beveled. Element 33: wherein the distal surface and the one or more elements are arranged radially outside the annular wall. Element 34: wherein the standoff feature exhibits an inner diameter larger that is the same as an inner diameter of the interior. Element 35: wherein the standoff feature provides a beveled distal surface that leads into the interior. Element 36: wherein the standoff feature comprises an undulating feature that provides a plurality of lobes angularly spaced from each other about a circumference of the body at the distal surface. Element 37: wherein each lobe extends a distance beyond the distal surface, and wherein the distance ranges between about 0.1 mm and about 5 mm. Element 38: wherein at least one of the one or more elements interposes angularly adjacent lobes of the plurality of lobes. Element 39: wherein at least one of the one or more lobes is curved and thereby provides a smooth and curved transition between the distal surface and the at least one of the one or more lobes. Element 40: wherein at least one of the one or more lobes provides a beveled distal surface that leads into the interior. Element 41: wherein the plurality of lobes extends substantially parallel to a central axis of the body.

By way of non-limiting example, exemplary combinations applicable to A, B, C, and D include: Element 4 with Element 5, Element 4 with Element 6, Element 6 with Element 7, Element 7 with Element 8, Element 7 with Element 9, Element 7 with Element 19,Element 7 with Element 11, Element 12 with Element 13, Element 19 with Element 20,Element 27 with Element 28, Element 27 with Element 29, Element 27 with Element 30,Element 30 with Element 31, Element 31 with element 32, Element 27 with Element 33,Element 33 with Element 34, Element 27 with Element 35, Element 36 with Element 37, Element 36 with Element 38, Element 36 with Element 39, Element 36 with Element 40, and Element 36 with Element 41.

The terminology used herein is for the purpose of describing particular embodiments only and is not intended to be limiting of the invention. As used herein, for example, the singular forms “a,” “an,” and “the” are intended to include the plural forms as well, unless the context clearly indicates otherwise. It will be further understood that the terms “contains”, “containing”, “includes”, “including,” “comprises”, and/or “comprising,” and variations thereof, when used in this specification, specify the presence of stated features, integers, steps, operations, elements, and/or components, but do not preclude the presence or addition of one or more other features, integers, steps, operations, elements, components, and/or groups thereof.

Terms of orientation are used herein merely for purposes of convention and referencing and are not to be construed as limiting. However, it is recognized these terms could be used with reference to an operator or user. Accordingly, no limitations are implied or to be inferred. In addition, the use of ordinal numbers (e.g., first, second, third, etc.) is for distinction and not counting. For example, the use of “third” does not imply there must be a corresponding “first” or “second.” Also, if used herein, the terms “coupled” or “coupled to” or “connected” or “connected to” or “attached” or “attached to” may indicate establishing either a direct or indirect connection, and is not limited to either unless expressly referenced as such.

The use of directional terms such as above, below, upper, lower, upward, downward, left, right, and the like are used in relation to the illustrative embodiments as they are depicted in the figures, the upward direction being toward the top of the corresponding figure and the downward direction being toward the bottom of the corresponding figure.

While the disclosure has described several exemplary embodiments, it will be understood by those skilled in the art that various changes can be made, and equivalents can be substituted for elements thereof, without departing from the scope of the invention. In addition, many modifications will be appreciated by those skilled in the art to adapt a particular instrument, situation, or material to embodiments of the disclosure without departing from the essential scope thereof. Therefore, it is intended that the invention not be limited to the particular embodiments disclosed, or to the best mode contemplated for carrying out this invention, but that the invention will include all embodiments falling within the scope of the appended claims. Moreover, reference in the appended claims to an apparatus or system or a component of an apparatus or system being adapted to, arranged to, capable of, configured to, enabled to, operable to, or operative to perform a particular function encompasses that apparatus, system, or component, whether or not it or that particular function is activated, turned on, or unlocked, as long as that apparatus, system, or component is so adapted, arranged, capable, configured, enabled, operable, or operative.

The invention claimed is:

1. A catheter, comprising:
an elongate shaft having opposing proximal and distal ends;
a tip structure coupled to the distal end of the elongate shaft and including:
an end surface at a distal end of the tip structure;
an inner surface defining a lumen terminating at the distal end of the tip structure; and
an outer surface opposite the inner surface; and
one or more elements arranged on the inner surface of the tip structure and configured to propagate shockwaves to fragment an object.

2. The catheter of claim 1, wherein the one or more elements are selected from the group consisting of an electrode pair, a laser element, and a piezoelectric element.

3. The catheter of claim 1, wherein the one or more elements comprise one or more first elements, the catheter further comprising one or more second elements positioned on an inner surface of the elongate shaft and configured to propagate shockwaves to fragment one or more objects located within the elongate shaft.

4. The catheter of claim 1, wherein the inner surface of the tip structure includes:
a first inner surface portion extending proximally from the distal end of the tip structure; and a second inner surface portion extending from the first inner surface portion proximally to the proximal end,
wherein the one or more elements are arranged on the first inner surface portion.

5. The catheter of claim 4, wherein the one or more elements comprise one or more first elements, the catheter further comprising one or more second elements arranged on the second inner surface portion to propagate shockwaves to fragment the object.

6. The catheter of claim 5, wherein the one or more first elements comprise a first array of elements, the catheter further comprising a second array of elements arranged on the outer surface of the tip structure to propagate shockwaves.

7. The catheter of claim 1, further comprising a standoff feature at the distal end extending distally from a portion of the inner surface where the one or more elements are arranged to form a separation gap between the one or more elements and the object to be fragmented.

8. The catheter of claim 7, wherein the tip structure further comprising a body, wherein the standoff feature extends from a distal end of the body, and wherein the one or more elements are arranged on a distal surface of the body.

9. A catheter, comprising:
an elongate shaft having opposing proximal and distal ends;
a tip structure coupled to the distal end of the elongate shaft and including an end surface at a distal end of the tip structure, the tip structure further providing:
an inner surface defining a lumen and terminating at the distal end of the tip structure, wherein the inner surface includes a first inner surface portion extending from the distal end of the tip structure and exhibiting a variable inner diameter and a second inner surface portion extending from the first inner surface portion and having a substantially constant inner diameter;
an outer surface opposite the inner surface; and
one or more elements arranged on the second inner surface portion and configured to propagate shockwaves to fragment an object located within the tip structure.

10. The catheter of claim 9, wherein the one or more elements comprise one or more first elements, the catheter further comprising one or more second elements arranged on an inner surface of the elongate shaft and being configured to propagate shockwaves to fragment one or more objects located within the elongate shaft.

11. The catheter of claim 9, wherein the one or more elements comprise one or more first elements, the catheter further comprising one or more second elements arranged on the first inner surface portion configured to propagate shockwaves.

12. The catheter of claim 11, further comprising one or more third elements arranged on the outer surface of the tip structure to propagate shockwaves away from the outer surface.

13. A tip structure for a catheter, comprising:
a cylindrical body having opposing distal and proximal ends and defining an interior that extends between the distal and proximal ends;
a distal surface provided at the distal end;
one or more elements mounted to the cylindrical body at the distal surface and configured to generate shockwaves to fragment an object; and
a standoff feature extending distally from the distal surface to form a separation gap between the one or more elements and the object to be fragmented.

14. The tip structure of claim 13, wherein the distal surface is planar and extends substantially perpendicular to a central axis of the body.

15. The tip structure of claim 13, wherein the standoff feature forms an integral part and extension of the body.

16. The tip structure of claim 13, wherein the standoff feature comprises an annular wall extending distally from the distal surface.

17. The tip structure of claim 16, wherein the standoff feature exhibits an inner diameter larger than an inner diameter of the interior, and wherein the distal surface and the one or more elements are positioned within the inner diameter of the standoff feature.

18. The tip structure of claim 16, wherein the distal surface and the one or more elements are arranged radially outside the annular wall.

19. The tip structure of claim 13, wherein the standoff feature comprises an undulating feature that provides a plurality of lobes angularly spaced from each other about a circumference of the body at the distal surface.

20. The tip structure of claim 19, wherein at least one of the one or more elements interposes angularly adjacent lobes of the plurality of lobes.
